SEQUENCE LISTING (3 sequences)

SEQ ID NO: 1 - DNA, 51 bp, artificial sequence
  example for hairpin sequence (see Fig. 6)
ctagcctgactgcatgctcttcgaatgcactgagcgcattcgaagagcatg

SEQ ID NO: 2 - DNA, 52 bp, artificial sequence
  example for hairpin sequence (see Fig. 6)
ctagcctgactgcatgctcttcgaatgcactgagcgcattcgaagagcatgc

SEQ ID NO: 3 - DNA, 53 bp, artificial sequence
  example for hairpin sequence (see -Fig. 6)
ctagcctgactgcatgctcttcgaatgcactgagcgcattcgaagagcatgca

(19) United States
(12) Patent Application Publication
(10) Pub. No.: US 2008/0038718 A1
(43) Pub. Date: Feb. 14, 2008

(54) Method For Parallelly Sequencing A Nucleic Acid Mixture By Using a Continuous Flow System

(76) Inventor: Achim Fischer, Heidelberg (DE)

Correspondence Address: FOLEY AND LARDNER LLP;SUITE 500, 3000 K STREET NW, WASHINGTON, DC, 20007, US

(21) Appl. No.: 10/515,954

(22) PCT Filed: Aug. 9, 2002
(86) PCT No.: PCT/EP02/08918

§ 371 Date: Apr. 19, 2007

(30) Foreign Application Priority Data
May 29, 2002 (DE) 102 24 339.5

(51) Int. Cl. C12Q 1/68; C12M 1/00

(52) U.S. Cl. 435/6; 435/283.1

(57) ABSTRACT

The invention relates to a method for the parallel sequencing of nucleic acids, comprising the steps:
1. Providing a porous support possessing areas distinguished by immobilized nucleic acid molecules,
2. Inserting the support of step (1) into a flow through arrangement,
3. simultaneously determining at least a part of the nucleotide sequence of at least a part of the nucleic acid molecules.

The invention relates to a method of simultaneously sequencing a plurality of different nucleic acid molecules as well as a porous support useful for carrying out the method.

Within biological analytics, sequence analysis of nucleic acids is an important method. This method allows the precise base sequence of the DNA or RNA molecules of interest, respectively, to be determined. Knowing this base sequence allows for, e.g., identification of certain genes or transcripts (i.e., the messenger RNA molecules derived from the genes), detection of mutations and polymorphisms, or even identification of organisms and viruses which can unambigously be recognized by certain nucleic acid molecules. Usually, nucleic acid sequencing is performed according to the chain termination method (Sanger et al. (1977), PNAS 74, 5463-5467). Thus, enzymatic complementation of a singlestranded nucleic acid to a doublestranded nucleic acid is performed. A so-called primer (usually a synthetic oligonucleotide), hybridized to said singlestranded nucleic acid, is elongated after addition of DNA polymerase and nucleotides. A small percentage of chain-terminating nucleotides (“chain terminators”), which upon incorporation into the growing strand inhibit further elongation, results in accumulation of partial strands distinguished by a known end which is determined by the respective chain terminator. The mixture of strands differing in length is then resolved by gel electrophoresis according to size. The nucleotide sequence of the unknown strand can then be derived from the obtained band patterns. A major disadvantage of this procedure is the great effort required concerning instrumentation which limits the achievable throughput. Each sequencing reaction requires (provided chain terminators labeled with four different fluorophores are used) at least one lane on a slab gel or, if capillary electrophoresis is used, at least one capillary. Using the currently most-advanced, commercially available sequencing machines, the resulting effort limits the number of sequencing reactions which can be processed in parallel to 384. What's more, reagents required for each sequencing reaction cause relatively high costs. A further disadvantage is the limitation of read length, i.e., the number of correctly identified bases per sequencing run, by the gel system's resolution. An alternative method for sequencing, sequencing by mass spectrometry, is faster and, thus, allows processing of more samples within the same time frame. However, sequencing by mass spectrometry is limited to relatively small DNA molecules, e.g., of a length of 40-50 bases. Still another sequencing technology, sequencing by hybridization (SBH, see Drmanac et al., Science 260 (1993), 1649-1652), base sequences are identified by specific hybridization of unknown samples with known oligonucleotides. Said known oligonucleotides are attached, in a complex array, to a support, hybridization with the labeled nucleic acid to be sequenced is performed, and the hybridizing oligonucleotides are determined. From the information as to which oligonucleotides hybridized to the unknown nucleic acid and from their sequence the sequence of the unknown nucleic acid can be determined. A disadvantage of the SBH technology is the fact that optimal hybridization conditions for oligonucleotides cannot be predicted exactly. Thus, it is not possible to design a large set of oligonucleotides comprising, on one hand, all possible sequence variants (defined by their, given length) and, on the other hand, sharing exactly the same optimal hybridization conditions. As a consequence, unspecific hybridization results in sequencing errors. Moreover, SBH can't be used for repetitive regions of nucleic acids to be sequenced.

Besides analysis of the level of expression of known genes such as made possible by dot blot hybridization, Northern hybridization or quantitative PCR, respectively, there are also technologies known from the art allowing for de novo identification of unknown genes differentially expressed between different biological samples. One strategy for gene expression analysis of this sort is the quantification of discrete sequence units. Such units can be so-called expressed sequence tags, ESTs. If a sufficient number of clones from cDNA libraries derived from samples to be compared is sequenced, identical sequences can be detected and counted, and the relative abundances of these sequences can be compared between different samples (see Lee et al., PNAS 92 (1995), 8303-8307). Different relative abundances of a particular sequence indicates differential expression of the corresponding transcript. However, this procedure requires considerable efforts since even for the quantification of the most abundant transcripts, sequencing of thousands of clones is required. On the other hand, unambiguous identification of a transcript usually requires only a short stretch of sequence of about 13-20 base pairs. The method called “serial analysis of gene expression” (SAGE; Velculescu et al., Science 270 (1995), 484-487) makes use of this fact. Here, short stretches of DNA (called “tags”) are concatenated, cloned,.and the resulting clones are sequenced. In this way it is possible to determine about 20 tags with a single sequencing reaction. However, this technology is still not yet very powerful since even for quantification of the most abundant transcripts many conventional sequencing reactions have to be performed and analyzed. Due to the considerable effort required, reliable quantification of rare transcripts using SAGE is very difficult.

A method for parallel sequencing of nucleic acid tags is disclosed in WO 98/44151. A surface is coated with PCR primers, followed by amplification at the surface of nucleic acid molecules to be sequenced. Thus, single nucleic acid molecules serving as template give rise to “DNA colonies” or “DNA islands” of identical molecules, respectively, which subsequently can be sequenced. The use of two immobilized PCR primers for amplification, which is already known from U.S. Pat. No. 5,641,658, has severe disadvantages: Firstly, the topology of nucleic acid strands attached to and bent back to the surface is unfavorable for the process of primer extension, resulting in a very low amplification efficiency (Adessi et al., Nucleic Acids Res 28 (2000), e87). To compensate for this, an unusually high number of amplification cycles is required, which due to the considerable thermal stress the nucleic acid molecules are subjected to can lead to strand breaks and partial detachment of primers from the surface. Secondly, amplification products are not accessible to any further analysis by hybridization or sequencing as long as they are attached to the surface at both ends. Thus, a single-sided detachment of nucleic acid molecules is required. In other words, before analysis can start, selectively one of the two ends has to be detached from the surface. This detachment in turn, which may be achieved by incubation with an appropriate restriction enzyme as described in WO 98/44151, is not unproblematic since restriction enzymes frequently can't cut nucleic acids bound to a solid support to completion. A further disadvantage of this method is the fact that single nucleic acid islands which might be of interest, e.g., due to their hybridization behavior, cannot be recovered and identified. Furthermore, no possibility of sequencing more than very short stretches (about 15-20 bases) of the island-forming nucleic acids is disclosed.

The methods for sequencing nucleic acids known from the art are distinguished by one or more of the following disadvantages:
they are rather limited in allowing the parallel processing of individual sequencing reactions.
they result in high costs per sequencing reaction.
they require relatively large amounts of the nucleic acid to be sequenced.
they are only appropriate to determining the sequence of short stretches of DNA and require considerable efforts in terms of instrumentation.

WO 01/61054 discloses a method for simultaneous performance of a plurality of micro-volume reactions on a substrate distinguished by a plurality of holes. Reaction chambers are formed by the holes, and the liquid in the reaction chambers is retained by the surface tension in the holes. Among others, micro-volume reactions can be sequencing reactions. Performing cycle sequencing according to the chain termination method, labeled singlestranded nucleic acids differing in length are generated and subsequently separated according to their size, e.g., by capillary electrophoresis or gel electrophoresis. The sequence can be concluded from the band patterns.

This method has the disadvantage that in any case a separating step has to be introduced to obtain sequences. In the event that separation of the labeled nucleic acids is performed by gel electrophoresis, there are handling problems referring to the transfer of the reaction volumes to the gel as well as problems referring to the sensitivity of detection as a consequence of the small volumes. In the event that separation is to be performed by capillary electrophoresis, there are insulation problems caused by the high voltages to be applied. Besides, correct alignment of the substrate along the electric flux lines is difficult. Finally, the sequencing method based on Taq polymerase results in significantly more sequencing errors than other sequencing methods not dependent on thermostable polymerases.

It is the object of the invention to provide a method which does not have the disadvantages of the prior art.

The object of the invention is achieved by a method for the massively parallel sequencing of nucleic acids, comprising the steps:
(1) Providing a porous support, possessing areas distinguished by immobilized nucleic acid molecules,
(2) Inserting the support of step (1) into a flow through arrangement,
(3) simultaneously determining at least a part of the nucleotide sequence of at least a part of the nucleic acid molecules.

The porous support is a solid body comprising hollow spaces. The support may be gel-like, however, preferably it is solid. The hollow spaces can be filled with a liquid or a gas, in particular they can be permeated by the liquid or gaseous phase surrounding the support. The hollow spaces can have any regular or irregular shape. The hollow spaces of a porous support can be of essentially the same size and/or shape, however, they can be of different size and/or shape as well. In any case, the porous support shall be an open-pored solid body, thus, the hollow spaces shall communicate with each other and/or with the surrounding liquid or gaseous medium at least in part. Here, “to communicate” means the possibility of exchanging matter, e.g., by diffusion, convection or active transport processes such as those which can be achieved by generating a pressure difference. This is not meant to exclude the possibility that the porous support is not one, but several solid bodies, e.g., a packing having hollow spaces of solid, identical or non-identical particles. The porous support's material can be chosen largely at will, as long as the requirements concerning structure (see above), wettability, resistance to solvents, resistance to temperature, compatibility with the steps to be performed of nucleic acid sequencing etc. are met. The porous support can consist of a polymer, glass, silicon, or another metallic, semi-metallic or nonmetallic substance. It is also conceivable to manufacture porous supports from more than one material; e.g., by co-polymerization of different monomers, by sintering of particles consisting of different materials, by coating the hollow spaces' walls of a porous solid body with one or more other arbitrary substances, or by addition of filling material to a matrix mediating strength. Most of the time, the porous supports employed for achieving the object of the invention are formed regularly, in particular flat, preferably having two parallel even surfaces, as to allow assigning to a support a top side and a bottom side, respectively. To a great extent, the support can be of any thickness, however a thickness between 50 μm and 20 mm, in particular between 300 μm and 1 mm, is preferred. It is particularly preferred to use a porous support distinguished by channels, in particular supports, whose channels are delimited on one side by the top side and on one side by the bottom side of the support, thus, whose top side and bottom side communicate with each other via at least a part of the channels. It is preferred that the channels are essentially in parallel to each other and/or at right angles to or about at right angles to the support's top side and/or the support's bottom side, as well as that the channels are cylindrical and essentially have the same diameter, thus, do not deviate more than, e.g., 10% or not more than 50%, respectively, from an average diameter. As a rule, the channels' diameter shall not exceed 200 μm. In a preferred embodiment, the channels' diameter is at most 100 μm, at most 30 μm, at most 10 μm, at most 3 μm, at most 1 μm or at most 0.3 μm. In a particularly preferred embodiment, the channels' diameter is between 0.5 μm and 50 μm, particularly between 5 μm and 25 μm. The use of so-called “glass capillary arrays” (“GCAs”, Burle Electro-Optics, Inc., Sturbridge, Mass., U.S.A.) or of “nanochannel glass” (Tonucci et al., Science 258:783-5 (1992)) is very particularly preferred, as is the use of porous silicon wafers.

The areas of step (1) can comprise one or more hollow spaces, in particular one or more channels. An area is not preferentially defined by a property of the porous support itself (such as, e.g., a special form etc., although one such shall not be excluded), but rather by the immobilized nucleic acid molecules' inhomogenous distribution. For example, a particular channel can contain a specified sort of nucleic acid molecules which occur immobilized to its wall, while neighbor channels adjoining this channel do not contain said sort of nucleic acid molecules; this channel then makes up its own area. However, several neighboring channels (i.e., channels adjoining each other) can also contain the same sort of nucleic acid molecules immobilized along their wall; then an area is made up of the entirety of these said channels.

In any case, nucleic acid molecules immobilized within an area shall of course have essentially identical sequences to allow for determination of their sequence; however, here nucleic acid molecules of different sequence not participating in the process of sequence determination do not do any harm. “To have identical sequences” refers to nucleic acid molecule-single strands, their “opposite strands” of essentially complementary sequence, as well as the double strands formed from strand and opposite strand. Nucleic acid molecules having only partially identical sequences, which, e.g., can have at least one identical and at least one non-identical sequence portion, are also not harmful as long as the sequence determination predominantly relates to the identical sequence portion.

Generating the areas carrying immobilized nucleic acid molecules is possible by a transfer, followed by an immobilization, of pre-formed solutions of nucleic acid molecules to the porous support, as well as by in situ-generation of numerous nucleic acid molecules having identical sequences by amplification of in each case one starting molecule at the site of the support, in particular within the support's hollow spaces, in the course of which an immobilization can take place during and/or after the amplification. Of course, it is further possible to first amplify nucleic acid molecules transferred to the support in the form of of pre-formed solutions of nucleic acid molecules and immobilize then only thereafter. In any case, the pre-formed solutions of nucleic acid molecules preferentially are a collection of solutions of one sort of nucleic acid molecule in each case, which can be deposited in appropriate containers such as, e.g., microtiter plates. The nucleic acid molecules can be generated, e.g., by processing of genomic DNA or of mRNA. In a preferred embodiment, genomic DANN is cut with one or more, in most cases frequently cutting restriction endonucleases, the resulting fragments are cloned, and DNA isolated from the clones (e.g., phage clones, bacterial clones, or yeast clones) or in vitro generated copies thereof are deposited as “genomic library”. In a further preferred embodiment, mRNA is transcribed to first strand cDNA, this is converted to doublestranded cDNA, and the process as described for genomic DNA is continued. The transfer of the solutions of nucleic acid molecules to the porous support can take place by methods according to the state of the art for production of DNA arrays, e.g., by applying appropriate volumes of liquid (e.g., between 1 nl and 100 nl), with the help of specially formed needles (“pins”), capillaries, or by use of the ink jet technology (e.g., piezo technology or bubble jet technology), to the surface of the porous support. Usually, the applied liquid is, by capillary effect, absorbed by the support, such that the respective area containing certain nucleic acid molecules results from the extent of all those hollow spaces of the support filled (i.e., whose walls have been wetted) by the transferred drop of liquid (or, where appropriate, several drops of liquid). In a special case, said area consists of only one single hollow space, e.g., one single capillary. However, in most cases an area will comprise several adjoining hollow spaces/capillaries.

In case it is intended to generate the immobilized areas, as an alternative to filling the porous support's hollow spaces with pre-formed solutions of nucleic acid molecules, by amplification of individual nucleic acid molecules to “clones”, i.e., accumulations of nucleic acid molecules having essentially the same sequence, localized at different areas of the support, usually at first an appropriately diluted solution of the mixture of nucleic acid molecules to be amplified in an appropriate amplification mixture is prepared, which contains, apart from the “template” molecules to be amplified, the components required for performing the amplification reaction(s), in particular aqueous buffer, nucleotide triphosphates (dNTPs), ions, at least one polymerase as well as in many cases amplification primers. This solution is contacted with the porous support in such a way that the support's hollow spaces are filled partially or completely, respectively, with solution. In a preferred embodiment, the template nucleic acid molecules' concentration is chosen such that in the plurality of hollow spaces there is at most one amplifiable template nucleic acid molecule. In a further preferred embodiment, there are on average, by way of calculation, at most 0.5 amplifiable nucleic acid molecules in a hollow space. In another preferred embodiment, there are on average, by way of calculation, at most 0.2 amplifiable nucleic acid molecules in a hollow space. In still another preferred embodiment, there are on average, by way of calculation, between about 0.1 and 0.02 amplifiable nucleic acid molecules in a hollow space. If, after introduction of the amplification solution into the porous support's hollow spaces, amplification conditions are established, in those hollow spaces which contain an amplifiable template molecule, copies of the very same are produced. The generation of at least 10⁶ copies of a template molecule is preferred, the generation of at least 10⁷ copies or at least 10⁸ copies is particularly preferred. Here, amplification can take place by use of any appropriate, isothermal or non-isothermal method such as PCR, NASBA, RNA amplification, rolling circle replication, or replication via Q beta replicase. As a result of the amplification, many hollow spaces of the porous support in each case contain numerous, preferably at least 10⁶, at least 10⁷ or at least 10⁸ copies of in each case one nucleic acid molecule, in the course of which different hollow spaces typically contain at least in part copies of nucleic acid molecules having a different sequence. As nucleic acid molecules to be amplified, e.g., restriction fragments obtained from genomic DNA or cDNA, or un-shortened cDNA molecules (so-called full size cDNAs) can be employed as well. In a preferred embodiment, these restriction fragments or molecules, respectively, are provided, at one end or at both ends, with “universal” primer binding sites common to several different molecules or preferably common to all molecules. This can happen by cloning in an appropriate vector, but also by attachment of “linkers”, i.e. doublestranded DNA molecules of a length of, e.g., between 15 bp and 50 bp. For performing the amplification reaction, it can be desired to reduce or even abolish completely the evaporation of water from the hollow spaces. This can be achieved by a number of different measures. For example, the porous support containing the amplification solution can be introduced into an atmosphere saturated by water vapor, or it can be contacted with a hydrophobic substance such as, e.g., paraffin oil or mineral oil. Further it is possible to contact the support on one side or on two sides with surfaces which tightly seal off with the support. These could consist, e.g., of glass or a polymer. In a preferred scheme for performing the amplification, the porous support containing the amplification solution is contacted on one side with a surface whose temperature is or can be controlled appropriately and covered on the other side with oil; then the temperature/temperatures appropriate to amplification are established. In a further preferred embodiment the porous support containing the amplification solution is dipped into an oil bath whose temperature is or can be controlled appropriately; then, the oil is adjusted to a temperature or temperatures, respectively, being appropriate to the amplification. In both cases it is possible, for performing nonisothermal amplification reactions, to effect appropriate temperature changes, where appropriate, in cyclic sequence.

In a modification of the method of the invention, the procedure is like in the above embodiment, but the nucleic acid molecules generated by amplification are not immobilized to the porous support's walls, but to a preferably planar surface contacted thereto. Then, the immobilization can be carried out during and/or after the amplification. After immobilization, the porous support is removed from the surface such that the immobilized nucleic acid molecules exist in the form of defined areas on the surface. Then, said nucleic acid molecules existing in areas on the surface are sequenced at least partially, e.g., according to one of the procedures mentioned below in (a) to (d). Thus, said modification relates to a method for the massively parallel sequencing of nucleic acids, comprising the steps:
(1) Providing a porous support,
(2) introducing an amplification mixture, containing amplifiable nucleic acid molecules, into the porous support's hollow spaces,
(3) performing an amplification of nucleic acid molecules in the porous support's hollow spaces,
(4) contacting the porous support with a surface, this step optionally having been carried out before performing the amplification,
(5) immobilization of at least a part of the amplified nucleic acid molecules of step (3) to the surface,
(6) simultaneous determination of at least a part of the nucleotide sequence of at least a part of the nucleic acid molecules immobilized to the surface.

The immobilization of the nucleic acid molecules may be carried out according to methods known from the art, it being preferred that the molecules are terminally immobilized, i.e., via their 3′-end or via their 5′-end. The immobilization shall be irreversible, i.e. that, under the conditions required for determination of the nucleotide sequence in step (3) (temperature, ionic strength, enzymatic activity, etc.), at most a part of the immobilized molecules, preferentially at most 10% or at most 50%, detaches from the porous support. Particularly preferred is a detachment of at most 5% or at most 10% of the immobilized nucleic acid molecules during determination of the nucleic acid sequence. The immobilization can be mediated by non-covalent interactions, e.g., the nucleic acid molecules to be immobilized can carry biotin groups. In this case, the porous support could be coated with avidin or streptavidin, respectively, such that a binding of biotin-modified nucleic acid molecules to the coated support can occur. However, immobilization of nucleic acid molecules by covalent interactions is preferred. For this, usually appropriately modified nucleic acid molecules are employed, e.g., nucleic acid molecules containing a 5′- or 3′-terminal amino group (“amino modifier” see Glen Research, Sterling, Virginia 20164: Catalog 2002, p. 56; f.), a terminal thiol group, a terminal phosphate group, an acrydite group, a carboxy-dT group, or another reactive group. If desired, there may be any sort of spacer or linker, e.g., an oligoethyleneglycol spacer or a cleavable group such as, e.g., a dithiol group or a photolytically cleavable nitrobenzyl group, respectively, between the reactive group mediating immobilization and the nucleic acid molecule. Such cleavable groups allow, if desired, after adjusting appropriate conditions, recovery of immobilized nucleic acid molecules by detachment from the porous support. Of course, the groups mediating the immobilization may also be located at other positions of the nucleic acid molecules, e.g., as side chains of the nucleotide bases as well as at the nucleic acid molecules' termini. The latter would be conceivable, e.g., by incorporation of aminoallyl dUTP or biotin dUTP during generation of the nucleic acid molecules. In any case, at first support and nucleic acid molecules are prepared in an appropriate way, such that support and nucleic acid molecules to be immobilized in each case possess one partner of a specific binding pair consisting of two partners, and such that immobilization of nucleic acid molecules by binding of both partners to each other can occur. In this connection, it shall of course not be excluded that further components can also be involved in the binding of both partners of the binding pair. There are numerous methods known from the art for the immobilization of biomolecules; examples are given in, e.g., Nucleic Acids Res. 22, 5456-65 (1994), and Nucleic Acids Res. 27, 1970-77 (1999). A further goal to be achieved in the course of immobilization is an appropriately high density of nucleic acid molecules on the surface, which shall guarantee a sufficient signal intensity during the process of sequencing. When fluorophores known from molecular biological applications such as, e.g., FITC, FAM, Cy3 or Cy5 are employed and the nucleic acid molecules to be sequenced are labeled by one fluorophore each, the density of nucleic acid molecules on the surface preferentially amounts to at least 10 molecules/μm², at least 100 molecules/μm², at least 1,000 molecules/μm², or at least 10,000 molecules/μm².

Immobilization of nucleic acid molecules generated by amplification can take place during or only after amplification. During amplification, immobilization is possible, e.g., by employing, in the course of a method for amplification based on primer extension such as PCR, in addition to primers present in solution, primers for their part already immobilized to the hollow spaces inner walls (or even exclusively immobilized primers, such as described, e.g., in WO 96/04404), which then can hybridize to the single stranded template molecules as well and subsequently be incorporated by primer extension. Thus, according to the spirit of the present invention, immobilization of a nucleic acid molecule present in solution can also mean synthesis of an opposite strand molecule complementary hereto, by extension of a primer hybridized to the dissolved molecule, thus, a “transcription” of the molecule from the liquid to the solid phase.

A feature of the flow through arrangement of step 2 is two spaces connected with each other via the porous support, such that liquids can flow from one of these spaces through the porous support to the other of the two spaces. A further feature of the arrangement can be a means to generate a pressure difference between both spaces, such that active transport of liquids is possible. Here, liquids means solutions, usually aqueous solutions, containing in particular the reagents required for determination of the nucleotide sequence in step (3). In a preferred embodiment, the flow through arrangement is designed such that observation of processes involving the nucleic acid molecules immobilized to the porous support is possible. “Observation” means here registration of changes of selected properties of the areas comprising immobilized nucleic acid molecules, such as conductivity, capacity, refraction index, luminescence, fluorescence, absorption of radiation, etc. Particularly preferred is the observation of optical phenomena, in particular luminescence or fluorescence, respectively. Accordingly, a further feature of the apparatus employed for carrying out the method of the invention is at least one detector allowing said registration, thus, in particular an optical detector able to detect light in the infrared, visible and/or ultraviolet range. The detector can be a device known from confocal microscopy. In an alternative embodiment, the detector is a CCD camera with an optical system allowing for observation, with sufficient resolution, on or within the porous support. Preferentially the area of the porous support projected to one pixel of the CCD camera has a size of at most 100 μm×100 μm, particularly a size of at most 10 μm×10 μm or of at most 2 μm×2 μm. If it is intended that the sequencing process is observed by means of fluorescently labeled molecules, a further feature of the apparatus employed for carrying out the method of the invention is at least one source of radiation appropriate to inducing fluorescence, preferentially a source of monochromatic light, in particular a laser.

The simultaneous determination of at least a part of the immobilized nucleic acid molecules sequence may be carried out in any way, but preferentially is done by stepwise strand synthesis or strand degradation, respectively. “Stepwise” means that the nucleic acid strands modified in the course of sequencing are elongated or shortened simultaneously in each case by the same amount, i.e., by a known number of nucleotides, preferentially in each case by exactly one nucleotide. In each step, the identity of the respective nucleotide or sequence of nucleotides, respectively, is determined for the nucleic acid molecules immobilized to several, preferentially to all or essentially all areas of the porous support. Sequencing of the nucleic acid molecules may be carried out in the following ways, for example:
a) incorporation of nucleotide triphosphates (“ordinary” nucleotides, dNTPs), upon determination of reaction by-products,
b) incorporation of labeled nucleotides,
c) incorporation of reversibly labeled nucleotides,
d) incorporation of labeled reversible chain terminator nucleotides.

Upon sequencing according to procedure (a), the formation of pyrophosphate associated with the incorporation of nucleotide triphosphates can be measured. For this, it is possible to convert, by use of sulfurylase, the generated pyrophosphate into ATP, which, in turn, participates in a chemoluminescence reaction catalyzed by luciferase and can thus be detected (see Ronaghi et al., Analyt. Biochem. 242, 84-89 (1996)).

Upon sequencing according to procedure (b), a partial nucleic acid double strand containing the nucleic acid strand to be sequenced is incubated, under conditions favorable for a polymerase-catalyzed fill-in reaction, with in each case one sort of labeled nucleotide (e.g., labeled dATP). After washing away non-incorporated nucleotides, it is determined by means of detection of the label if any or how many nucleotides, respectively, have been incorporated (e.g., 1×A, 2×A, etc.). In the next step, it is incubated with a second sort of labeled nucleotide (e.g, labeled dCTP) and detected, then the same with a third (e.g., labeled dGTP), and finally with the fourth sort of nucleotide (e.g., labeled dTTP). Then, the cycle starts again by adding labeled nucleotide of the first sort. The signal intensities measured upon a detection result from in each case the sum of the signal intensity of the nucleotide incorporation of the nucleotide incorporation performed last and all the nucleotide incorporations performed beforehand.

In the procedure according to (c), the label of the incorporated nucleotides is deleted at appropriate times, e.g., after each nucleotide addition, after each cycle consisting of the sequential addition of all four different nucleotides, or of one or more repetitions thereof, respectively. Preferentially, this occurs by removal or modification of the labeling group or the labeling groups. For example, the labeling group can be bound to the respective nucleotide via a chemically, photochemically or enzymatically cleavable spacer, respectively, e.g., a spacer containing a disulfide group or a nitrobenzyl group. One possibility of modifying the labeling group would be, e.g., bleaching of a fluorescent dye, which would be feasible by sufficiently intense irradiation by a laser. The advantage of procedure (c) over (b) is that upon each measurement, in each case only a part of the incorporated nucleotides, ideally exclusively the in each case last incorporated nucleotide, is determined without the need to take into account the signal background of already incorporated nucleotides, which often amounts to several times that of the signal of interest.

The sequencing according to procedure (d) may be carried out such as described in U.S. Pat. No. 5,302,509, for example. Here, nucleotide-wise elongation of nucleic acid strands is achieved via employment of nucleotide triphosphates reversibly blocked at their 3′-OH group, which can be incorporated by polymerases into a growing DNA double strand, but which, after their incorporation, act as chain elongation terminators. When the blocking group is cleaved off, a free 3′-OH group is re-established, such that a next nucleotide can be incorporated. For example, Canard and Sarfati (Gene 148, 1-6 (1994)) describe reversibly blocked nucleotide triphosphates which, after their incorporation, can be identified by means of fluorescent labeling of the reversible protecting group.

If the immobilized nucleic acid molecules are to be sequenced in step (3) according to procedure (a), (b), (c), or (d), respectively, usually at least a part of the nucleic acid molecules will exist in at least a partially single stranded state. To be able to effect a sequence determination by incorporation of nucleotide building blocks into a growing strand according to the known base pairing rules, usually a so-called sequencing primer will be required, i.e., an oligo- or polynucleotide which is able to hybridize with the nucleic acid strand to be sequenced and which is present in a hybridized state such that it can, at its 3′-end, be elongated by a DNA polymerase, in the course of which the opposite strand complementary to the region to be sequenced is synthesized. Accordingly, as a step preceding the actual sequencing, frequently the immobilized nucleic acid molecule is; where appropriate, by removal of the opposite strand, converted to the single stranded state, and then an appropriate sequencing primer which is at least partially complementary to the nucleic acid molecule and which has a 3′-end extendable by a polymerase is hybridized with the nucleic acid molecule. In an alternative embodiment it is possible to let the nucleic acid molecule form a 3′-terminal hairpin structure or to attach such a structure to the nucleic acid molecule, which for their part can be elongated by a polymerase (see U.S. Pat. No. 5,798,210). In a particularly preferred embodiment, the nucleic acid molecules to be immobilized to the porous support and to be sequenced in step (3) are, at an end, preferentially at the other end which, after immobilization via one end, projects into the solution space, provided with a further single stranded or double stranded nucleic acid molecule which is folded back or is able to fold back, such as, e.g., a partially self-complementary oligonucleotide. It is also possible to attach to the nucleic acid molecule present in the double stranded state, prior to the immobilization, a “masked hairpin”, i.e., a doublestranded nucleic acid molecule containing an inverted repeat. Upon removal, after immobilization, of one of the two strands via denaturation, the opposite strand remaining at and attached, via its 5′-end, to the nucleic acid molecule to be sequenced, can then “fold back” and be elongated at its free 3′-end by a polymerase.

Of course, the above examples of sequencing methods per se known are not intended to exclude other sequencing methods being employed within the scope of the method of the invention.

The invention is characterized more closely by the following description.

The invention particularly concerns a method for parallel sequencing of nucleic acids, comprising the steps:
(i) Providing a monolithic porous support, having at least two sample chambers extending through the porous support, having at least an inlet and an outlet and possessing one or more surfaces to which nucleic acid molecules are immobilized, having a single stranded portion, the porous support having at least two distinguishable sites having nucleic acids of different sequence,
(ii) providing a solution containing one or more nucleotide compounds, selected from mononucleotides and oligonucleotides,
(iii) Introduction of the solution of step (ii) into the sample chambers of the porous support, by which binding of the nucleotide compounds to the immobilized nucleic acids' singlestranded portions and, thus, a mediated binding to the porous support is effected,
(iv) detection of amount and/or identity of the nucleotide compounds, at the at least two distinguishable sites of the porous support, bound, by means of the immobilized nucleic acids, indirectly to the porous support.

Steps (ii) to (iv) can be repeated once or several times, in the course of which, with each cycle, sequence information is obtained.

Introduction of the solution of step (ii) into the porous support's sample chambers is carried out in step (iii) by generating a current of the solution of step (ii) through the sample chambers.

In step (iv), the detection takes place as to whether the nucleotide compounds have been bound indirectly to the porous support. If the solution of step (ii) contains several nucleotide compounds, it is tested in step (iv) which nucleotide compound has been bound, i.e., its identity is determined. It is usually required to measure the amount of bound nucleotide compound, too, to be able to distinguish significant signals from background. Under certain conditions it is expedient to measure the amount more precisely. This is the case when, possibly, several nucleotide compounds can be bound in step (iii) to the immobilized nucleic acids' singlestranded portions, and when this allows conclusions to be drawn about the sequence, such as in the course of the sequencing by enzymatic strand extension with nucleotides without a chain termination group.

The porous support consists of a solid body having hollow spaces, which can be gel-like, but which preferentially is solid. The hollow spaces can be filled by liquid as well as filled by gas, particularly permeated by the liquid or gaseous phase surrounding the support. The hollow spaces can have any, regular or irregular, form. A porous support's hollow spaces can be of essentially the same shape and/or size, but they can be of uneven shape and/or size as well. Preferentially, the hollow spaces are dimensioned such that they, when filled with gas, are able to suck up liquid solutions by capillary forces or, when filled with liquid, are able to hold the liquids.

In any case, it shall refer to an open-pored solid body, i.e., the hollow spaces shall be able to communicate at least in part with the liquid or gaseous medium surrounding the solid body; further it is conceivable that the hollow spaces communicate with each other as well. Here, “to communicate” means the possibility of matter exchange, e.g., by diffusion, convection, or active transport processes such as can be achieved by establishing a pressure difference.

The porous support is a coherent solid body, a monolith, e.g, a packing having hollow spaces of solid, identical or non-identical particles or capillaries which are tightly, in particular covalently, connected. The porous support's material can be selected essentially arbitrarily, as long as the requirements concerning structure (see above), wettability, resistance to solvents, heat resistance, compatibility with the steps of nucleic acid sequence determination to be carried out, etc., are met. The porous support can be made of a polymer, e.g., copolymer of different monomers, of glass, of silicon, or another metallic, semi-metallic or non-metallic substance. It is also conceivable to manufacture porous supports of more than one material; e.g., by sintering of particles made of different materials, by coating the walls of a porous support's hollow spaces with one or several arbitrary different substances, or by addition of filling materials to a matrix mediating strength. Usually, the porous supports employed for achieving the object of the invention are formed regularly, particularly flat, preferentially having two parallel even faces. Usually, the porous support has surfaces opposed to each other, i.e., not adjoining each other, which preferentially are essentially parallel to each other, namely a first face and a second face, which preferentially represent the support's top side and its bottom side, respectively, and which preferentially (but not necessarily) are even. To a great extent, the support can have any thickness, however, a thickness between 50 μm and 20 mm, in particular between 300 μm and 1 mm, is preferred.

The porous support has at least two sample chambers, preferably more than 100, more than 10³, more than 10⁴ or 10⁵, more than 10⁶, particularly more than 10⁷, which are formed by the hollow spaces. A sample chamber extends through the whole porous support, thus, it runs through the porous support from outer surface to outer surface, and has in its lumen one or more surfaces and at least an inlet and an outlet, preferentially in each case one inlet and at least one or several outlets or in each case one outlet and at least one or several inlets. Usually, a sample chamber is distinguished by dimensions such that the contents, if they are liquid, are retained in the sample chamber by capillary forces.

Preferentially, the sample chambers have, along their axis, a regular, preferably round or hexagonal cross section. It is preferred that the sample chambers essentially have the same diameter, thus, they do not deviate by more than 50%, particularly not by more than 10% from an average diameter. However, the cross section can be irregular as well, such as will be the case with a porous support manufactured by a sintering procedure.

It is not excluded that various sample chambers are connected to each other. This is particularly true in case of porous supports manufactured by a sintering procedure. Use of such supports within the framework of this invention is possible as well. In this case a network exists which makes discrimination of individual sample chambers difficult. In this connection, it has to be borne in mind that the transport of matter along the axis of a sample chamber exceeds the transport of matter between two different chambers. The ratio of the transport of matter (by diffusion and convection in step (iii)) along a sample chamber's axis to the transport of matter between two different chambers amounts to at least 10, preferably at least 100, in particular at least 1000.

It is preferred that the sample chambers are running essentially in a straight line and are oriented within the porous support such that they have a preferred direction, which facilitates the generation of a current through the sample chambers in step (iii), since in this way it is ensured that a single current flows evenly through a plurality of sample chambers. A sample chamber's axis is defined by two points, the center of the inlet and the center of the outlet. If a sample chamber has several inlets or outlets, respectively, it thus has several axes. The sample chambers' axes preferentially form an angle of less than 30°, particularly less than 15°, above all less than 5°, an essentially parallel alignment being most preferred.

The nucleic acid molecules within a sample chamber represent a sequencing sample.

Preferably the nucleic acid molecules within the porous support are partitioned, i.e., the nucleic acid molecules immobilized within a sample chamber preferably have in regard of the singlestranded portion essentially the same sequence, thus, they have, within the scope of the following definition, preferably identical sequences. The term “to have identical sequences” is defined in the following and does not mean, within the scope of the invention, that the sequences have to be exactly the same, although, however, this usually will be the case. The term “to have identical sequences” takes into account that enzymatically generated nucleic acid molecules, due to faulty reproduction of a common template nucleic acid molecule, often have sequence errors representing a deviation from sequence identity, but being sufficiently rare so as not to prevent sequence determination. In this connection, however, nucleic acid molecules having a different sequence and not participating in sequence determination are not detrimental, i.e., they are left out of account when considering whether sequence identity is given. Also, sequence deviations of nucleic acid molecules having only partially identical sequences, e.g., nucleic acid molecules having at least an identical and at least a non-identical sequence part, can be left out of account as long as the sequence determination predominantly relates to the identical part of the sequence. Within the framework of the invention, the sequence determination preferentially is carried out according to the method of enzymatic strand extension. Provided intermolecular priming is employed, the sequence determination relates, according to the method of enzymatic strand extension, only to the portion of the sequence 3′ to the portion to which the sequencing primer employed for priming of the polymerase is binding. In case of intramolecular priming the nucleic acid only participates in the sequence determination when it is able to form a hairpin. In general it can be said that sequence determination according to the method of enzymatic strand elongation only relates to the portion of a nucleic acid which ranges, for as many bases as correspond to the maximum read length, in the 3′-direction from the boundary between doublestranded portion and singlestranded portion of the nucleic acid molecule.

In a preferred embodiment, the porous support comprises at least 100, above all at least 100, in particular at least 10³, at least 10⁴, at least 10⁵ or at least 10⁶, above all at least 10⁷ distinguishable sites possessing nucleic acids having a different sequence each, the sequence differences preferably referring to the nucleic acids' single stranded portions. Thus, the distinguishable sites in step (i) preferably possess nucleic acids with single stranded portions having different sequences. The term “distinguishable” refers to the detection carried out in step (iv), whose spatial resolution must allow for the identification of distinguishable sites. The sites according to the invention have a twodimensional or a threedimensional expansion, respectively.

Preferably, the distinguishable sites comprise in each case at least one sample chamber (more precisely, its surface(s)) on the porous support, but they can comprise several sample chambers as well. The sample chambers are formed by in each case at least one hollow space on the porous support.

In a preferred embodiment of the present invention, the sample chambers are designed as channels. The channels may be capillaries.

By forming at least an inlet and an outlet, the channels are opened to the support's first and second side, such that both sides of the support, e.g., the top and the bottom side, communicate via the channels. If the support's two sides are the top and bottom side, the channels are delimited on one side by the support's top side and on one side by the suppport's bottom side. Channels forming dead ends within the porous support do not correspond to the definition above, thus, the following explanations do not refer to channels of this kind. However, their presence in the porous support is not excluded.

Within the scope of the invention, the term “channels” includes those which are connected, thus, which communicate with each other, as well. This may lead to formation of a distinguishable site by several channels, each of which having their own inlet and outlet and which are connected with each other. Indeed this results in a reduction of resolution, i.e., of the maximum number of distinguishable sites which can be present within one unit area of the porous support according to step (i). However, this is not detrimental as long as the channels' diameter is sufficiently small to ensure a sufficient resolution despite the possibility of communication between some of the channels. An example of a porous support whose channels in part are connected with each other is the porous support called PamChip™-Array distributed by the company PamGene (PamGene, Burgemeester Loeffplein 70A, 5211 RX's-Hertogenbosch, NL).

It is preferred that the channels run essentially in a straight line and are oriented within the porous support such that they have a preferred direction, which facilitates generating of a current through the channels in step (iii) since in this way it is ensured that a single current flows evenly through a plurality of channels. Preferably, the channels' axes form an angle of less than 30°, particularly less than 15°, above all less than 5°, an essentially parallel alignment being most preferred.

It is further preferred that the channels run at right angles or approximately at right angles to the support's first and/or second side, which preferentially represent the porous support's top or bottom side, respectively.

It is further preferred that the channels are cylindrical or polygonal, in particular hexagonal. It is further preferred that they essentially have the same diameter, thus, they do not deviate, from an average diameter, by more than 50%, in particular not more than 10%. Usually, the channels' diameter should not exceed 200 μm. In preferred embodiments, the channels' diameter amounts to at most 100 μm, at most 30 μm, at most 10 μm, at most 3 μm, at most 1 μm or at most 0.3 μm. In a particularly preferred embodiment, the channels' diameter amounts to between 0.5 μm and 50 μm, particularly between 5 μm and 25 μm. Substrates disclosed by WO 99/34920 and WO 00/56456, which herewith are referred to, can also be employed as supports.

Particularly preferred is the use of so-called “glass capillary arrays” (“GCAs”, Burle Electro-Optics, Inc., Sturbridge, Mass., U.S.A.) or “nanochannel glass” (Tonucci et al., Science 258: 783-5 (1992)), respectively, or of porous silicon wafers.

In a preferred embodiment of the method of the invention, step (i) comprises the steps
(i-a0) Providing a monolithic porous support, having at least two sample chambers extending through the porous support, which have at least an inlet and an outlet and which possess one or more surfaces,
(i-a1) Soaking of the porous support with a nucleic acid solution containing at least two nucleic acid molecules having different sequences, such that at least two sample chambers are filled with the nucleic acid solution,
(i-a2) Amplifying the nucleic acid molecules within the sample chambers,
(i-a3) Immobilization of the nucleic acid molecules to the surfaces of the porous support's sample chambers, in the course of which steps (i-a2) and (i-a3) may be carried out simultaneously,
(i-a4) converting the nucleic acid molecules to a state where they have a singlestranded portion, in the course of which step (i-a4) may also be carried out before step (i-a3) or at the same time as step (i-a3), respectively.

Preferentially, the nucleic acid molecules in step (i-a4) have a singlestranded portion as well as a doublestranded portion.

In a further embodiment of the method of the invention, step (i) comprises the steps
(i-b0) Providing a monolithic porous support, having at least two sample chambers extending through the porous support, which have at least one inlet and one outlet and which possess one or more surfaces,
(i-b1) Transferring to different positions of the porous support at least two nucleic acid solutions containing in each case nucleic acid molecules having different sequences, such that at least two sample chambers are filled with the nucleic acid solutions,
(i-b2) Immobilization of the nucleic acid molecules to the surfaces of the porous support's sample chambers,
(i-b3) converting the nucleic acid molecules to a state where they have a singlestranded portion,
   in the course of which step (i-b3) may also be carried out before step (i-b2) or step (i-b1) or at the same time as one of these steps, respectively, and in the course of which step (i-b3) can be skipped when the nucleic acid molecules in step (i-b1) have a singlestranded portion.

Preferably the nucleic acid molecules in step (i-b3) have a singlestranded as well as a doublestranded portion.

The transferring in step (i-b1) preferably is carried out with the aid of pins, capillaries or by means of the ink jet technology.

The generation of immobilized nucleic acid molecules being positioned in the areas or, according to another version, in the channels, is possible by one of two measures:
(i-a) In situ-generation of numerous nucleic acid molecules having identical sequences by amplification of in each case one starting molecule within the porous support s sample chambers.
(i-b) Transferring to in each case different positions of the porous support at least two nucleic acid solutions containing in each case nucleic acid molecules having different sequences, followed by immobilization of the nucleic acids.

According to measure (i-a), the porous support is soaked, followed by amplification of the nucleic acids, with a solution containing at least two nucleic acid molecules having different sequences, thus, a mixture of different nucleic acids. First, an appropriately diluted solution of the mixture is produced, and the components required for a successful amplification added. Besides the nucleic acid molecules to be amplified (template molecules), this amplification solution particularly contains aqueous buffer, nucleotide triphosphates (dNTPs), ions, at least one polymerase, and, according to the method of amplification selected, amplification primers as well. The solution is contacted with the porous support such that the support's sample chambers are partially or completely filled with solution.

In this connection, the concentration of amplifiable nucleic acids is preferentially chosen such that the plurality of sample chambers (or channels, respectively) contains at most one (amplifiable) nucleic acid molecule. Non-amplifiable nucleic acids, particularly primers, are not considered. In this way, distinguishable sites are formed on the porous support which possess nucleic acids having different sequences. In a later step (i-a4), doublestranded nucleic acids are converted to a state in which they have a singlestranded portion. Usually, conversion of nucleic acid molecules to a state in which they have a singlestranded portion occurs by denaturation.

Usually, the porous support s distinguishable sites possessing nucleic acids having different sequences are such possessing nucleic acids with singlestranded portions having different sequences. This means that the sequence differences usually refer to the singlestranded portion. This is particularly valid for the method of sequencing by enzymatic strand extension. Here, distinguishability of the sites results from the fact that they are located on different sample chambers of the porous support and, thus, can be distinguished from each other upon detection. From the abovesaid it results that the distinguishable sites on the porous support are arranged statistically according to a random array.

In a further preferred embodiment, on average there are, by way of calculation, at most 0.5 amplifiable nucleic acid molecules within a sample chamber. In another preferred embodiment, there are on average, by way of calculation, at most 0.2 (amplifiable) nucleic acid molecules within a sample chamber. In still another preferred embodiment, there are on average, by way of calculation, between about 0.1 and 0.02 (amplifiable) nucleic acid molecules within a sample chamber. Non-amplifiable nucleic acids, particularly primers, are not considered.

Then, within those sample chambers containing an (amplifiable) nucleic acid molecule, an amplification of this molecule to numerous copies takes place. Generation of at least 10⁶ copies of a nucleic acid molecule is preferred, the generation of at least 10⁷ copies or at least 10⁸ copies is particularly preferred. Amplification can take place according to any appropriate, isothermal or non-isothermal method, such as PCR, NASBA, RNA amplification, rolling circle replication, or replication by use of Q beta replicase, PCR being preferred. As a result of amplification, the porous support's sample chambers, in which amplification had occured each contain a plurality of, preferentially at least 10⁶, at least 10⁷ or at least 10⁸ copies of in each case one nucleic acid molecule, different sample chambers typically containing at least in part copies of different nucleic acid molecules.

The nucleic acid molecules to be amplified in step (i-a2) can represent, e.g., restriction fragments obtained from genomic DNA or cDNA, or unshortened cDNA molecules (so-called full size cDNAs) as well.

In a preferred embodiment, these restriction fragments or molecules, respectively, are provided, at one end or at both ends, with “universal” primer binding sites common to several different molecules or, preferably, common to all molecules. This can take place by cloning into an appropriate vector, but also by attachment of linkers, i.e., doublestranded DNA molecules having a length of, e.g., between 15 bp and 50 bp. For performing the amplification it can be desired to reduce or to abolish completely the evaporation of water from the hollow spaces. This can be achieved by a number of different measures. For example, the porous support containing the amplification solution can be introduced to an atmosphere saturated by steam, or be contacted with a hydrophobic substance such as, e.g., paraffin oil or mineral oil. It is further possible to contact the support, on one side or on two sides, with surfaces tightly sealing off with the support. These could be made of, e.g., glass, metal, or a polymer.

In a preferred embodiment for performing the amplification, the porous support containing the amplification solution is contacted, on one side, with a surface whose temperature is or can be controlled appropriately and covered, on the other side, with oil; then the temperature/temperatures appropriate to amplification is/are adjusted.

In a further preferred arrangement, the porous support containing the amplification solution is immersed in an oil bath whose temperature is or can be controlled appropriately, respectively; then, the oil is adjusted to the temperature or temperatures appropriate to amplification. In both cases it is possible to carry out temperature changes, where appropriate, in a cyclic sequence, appropriate to performing non-isothermal amplification reactions.

The immobilization of nucleic acid molecules in step (i-a3) may be carried out during the amplification in step (i-a2) or after the amplification.

In a preferred embodiment of the method, amplification is carried out by PCR, and the immobilization during amplification takes place owing to the primers participating in the PCR reaction being immobilized to the sample chambers' surfaces. For this, in each case one primer or both primers of a primer pair are immobilized to the sample chambers' surfaces. If only one primer of a primer pair is immobilized, the other primer of the primer pair within the respective sample chamber exists in free solution, i.e., non-immobilized. Also, the primers of a primer pair can be immobilized only partially, i.e., only a certain percentage of a primer out of a primer pair is immobilized, whereas the other portion is not. This has the advantage that, when the concentrations of the nucleic acid to be amplified are low, amplification efficiency is comparatively good due to the availability of non-immobilized primers. This is for the benefit of the amplification's reliability. After several amplification cycles, an impoverishment of non-immobilized primers results, such that the amplification reaction continues by means of immobilized primers, which results in a lower amplification efficiency, but, however, in the immobilization of amplified nucleic acid molecules to the sample chambers'0 surfaces.

According to measure (i-b), transfer to in each case different positions of the porous support of at least two nucleic acid solutions occurs, each solution containing singlestranded or doublestranded nucleic acid molecules having different sequences. Then, a single nucleic acid solution of step (i-b1) in each case preferably contains only nucleic acid molecules having identical sequences according to the above definition. In particular, a single nucleic acid solution of step (i-b1) contains in each case only nucleic acid molecules having the same sequence.

The nucleic acid molecules transferred to the support are first, where appropriate, amplified there and immobilized only subsequently, or they are first immobilized and, where appropriate, amplified subsequently. However, amplification is optional. Whether an amplification makes sense depends on the amount of nucleic acid within the individual solutions of nucleic acid molecules applied to the support.

The nucleic acid solutions in step (i-b1) may be entire collections of solutions of nucleic acid molecules having identical sequences. In this connection, the solutions are contacted with the porous support in a way such that, where possible, no mixing of nucleic acid solutions each containing nucleic acid molecules having different sequences takes place. In this way, areas of nucleic acid molecules of one sort, which may comprise one or several sample chambers, are formed on the porous support. In this way, distinguishable sites possessing nucleic acids having different sequences are formed on the porous support. In a later step (i-b3), the nucleic acids are converted to a state in which they have a singlestranded portion.

As a rule, the distinguishable sites of the porous support possessing nucleic acids having different sequences are such that possess nucleic acids with singlestranded portions having different sequences. This means that, as a rule, the sequence differences refer to the singlestranded portion. This is particularly the case for the method of sequencing by enzymatic strand elongation. From what has been said it results that, according to measure (i-b), the distinguishable sites on the porous support are not arranged statistically. Rather, the position of each of the distinguishable sites can be chosen.

The nucleic acid solutions in step (i-b1) can be stored in appropriate containers such as, e.g., microtiter plates. The nucleic acid molecules can have been generated, e.g., by processing of genomic DNA or of mRNA.

In a preferred embodiment,. genomic DNA is cut with one or several, usually frequently cutting, restriction endonucleases, the resulting fragments are cloned, and the DNA isolated from the resulting clones (e.g., phage clones, bacterial clones, or yeast clones) or copies thereof generated in vitro are deposited as “genomic library”.

In a further preferred embodiment, mRNA is transcribed to first strand cDNA, this is converted to doublestranded cDNA, and the procedure as described for genomic DNA is continued, in the course of which the fragmentation with restriction endonucleases can be skipped.

The transfer of the nucleic acid solutions to the porous support in step (i-b1) may be carried out by applying to the surface of the porous support, according to methods for DNA array preparation known from the art, e.g., with the aid of specially formed needles (“pins”), capillaries, or by means of the ink jet technology (e.g., piezo technology or bubble jet technology), appropriate volumes of liquid (e.g., between 1 nl and 100 nl). Usually the liquid, which is preferably applied to a position on the porous support as one or several drops of liquid, is absorbed, by capillary effect, by the support, such as to form an area or distinguishable site where the nucleic acid molecules have the same sequence or have identical sequences according to the definition. In this connection, the area or distinguishable site of identical nucleic acids or nucleic acids having identical sequences covers all sample chambers of the support which have been filled, upon transfer of nucleic acid solution, by the drop or drops of liquid. In a special case, the area or distinguishable site of nucleic acids having identical sequences comprises only one single sample chamber, e.g., one single capillary.

The immobilization of nucleic acid molecules in step (i-a3) or (i-b2) can take place by means of methods known from the art; then it is preferred that the molecules are immobilized terminally, i.e., via their 3′-end or via their 5′-end, respectively. The immobilization shall be irreversible, i.e., under the conditions required for determination of the nucleotide sequence (temperature, ionic strength, enzymatic activity, etc.), at most a part of the immobilized molecules detaches from the porous support, preferably at most 10% or at most 50%. Particularly preferred is detachment, during determination of the nucleic acid sequence, of at most 5% or at most 1% of the immobilized nucleic acid molecules. The immobilization can be mediated by non-covalent interactions, e.g., the nucleic acid molecules to be immobilized can carry biotin groups. In this case the porous support could be coated with avidin or streptavidin, such that a binding of the biotin-modified nucleic acid molecules to the coated support can occur. However, immobilization of the nucleic acid molecules by covalent interactions is preferred. For this, usually appropriately modified nucleic acid molecules are employed, e.g., nucleic acid molecules containing a 5′- or 3′-terminal amino group (“amino modifier”, see Glen Research, Sterling, Virginia 20164: Catalog 2002, p. 56 f.), a terminal thiol group, a terminal phosphate group, an acrydite group, a carboxy-dT group, or another reactive group. If desired, there may be located between the reactive group mediating immobilization and the nucleic acid molecule any sort of spacer or linker, respectively, e.g., an oligoethylene glycol-spacer or a cleavable group such as, e.g., a dithiol group or a photolytically cleavable nitrobenzyl group. If desired, such cleavable groups allow, after adjustment of appropriate conditions, for recovery of immobilized nucleic acid molecules by detachment from the porous support. Of course, the groups mediating immobilization can be positioned, other than at the nucleic acid molecules' termini, at other positions within the nucleic acid molecules as well, e.g., as side chains at the nucleotide bases. For example, the latter would be conceivable by incorporation of aminoallyl-dUTP or biotin-dUTP, respectively, during generation of the nucleic acid molecules. In any case, support and nucleic acid molecules are first prepared in an appropriate way, such that support and nucleic acid molecules to be immobilized, respectively, each possess one partner of a specific binding pair consisting of two partners, and such that an immobilization of the nucleic acid molecules by binding of both partners to each other can occur. Of course, in this connection it shall not be excluded that there may be further components involved in the binding of the binding pair's two partners. There are many methods for immobilization of biomolecules known from the art; examples are given, e.g., in Nucleic Acids Res. 22, 5456-65 (1994) as well as in Nucleic Acids Res. 27, 1970-77 (1999). A further goal to be reached upon immobilization is an appropriately high density of nucleic acid molecules on the surface, which shall guarantee, during the sequencing process, a sufficient signal intensity. When fluorophores known from molecular biological applications such as, e.g., FITC, FAM, Cy3 or Cy5, respectively, are employed and the nucleic acid molecules to be sequenced are labeled by one fluorophore each, the density of nucleic acid molecules on the surface preferably amounts to at least 10 molecules/μm², at least 100 molecules/μm², at least 1,000 molecules/μm², or at least 10,000 molecules/μm². Concerning further examples of fluorescent dyes which may be employed, reference is made to the catalog of the company Molecular Probes, Eugene, Oreg., USA, 6th edition, 1996.

As already mentioned above in measure (i-a), the immobilization of nucleic acid molecules generated by amplification in step (i-a3) or, provided during measure (i-b) an amplification takes place, in step (i-b2), respectively, can take place during or only after the amplification. Immobilization during the amplification is possible, e.g., by employing, for an amplification method based on primer extension such as PCR, in addition to the primers present in solution, also primers for their part already immobilized to the hollow spaces inner walls (or even by employing immobilized primers exclusively such as, e.g., described in WO 96/04404), which then hybridize with the singlestranded template molecules as well and subsequently can be incorporated by primer extension. If the immobilization of nucleic acid molecules is carried out via primers immobilized to the sample chambers' surfaces, immobilization of only one primer of a primer pair is advantageous since in this way only one nucleic acid strand of a doublestranded nucleic acid molecule is immobilized, such that removal of the non-immobilized nucleic acid strand is facilitated, thus providing nucleic acid molecules with a singlestranded portion.

Accordingly, within the present invention's spirit, immobilization of a nucleic acid molecule present in solution also relates to, by extension of an immobilized primer hybridized to the dissolved molecule, synthesis of an opposite strand molecule complementary hereto, thus, in a way, a transcription of the molecule from the liquid to the solid phase.

A preferred embodiment of the invention relates to a method for the parallel sequencing of nucleic acids by enzymatic strand extension, in the course of which in step (i) the nucleic acid molecules having a doublestranded and a singlestranded portion and the nucleotide compounds in step (ii) are nucleotides and the solution also contains, besides the nucleotides, a strand extending enzyme, and in step (iv) the enzyme, with formation of hydrogen bonds, binds the nucleotides to the singlestranded portions of the immobilized nucleic acid molecules and, thus, indirectly to the porous support and incorporates them, at the boundary between the doublestranded portion and the singlestranded portion, into the immobilized nucleic acid molecules.

This preferred embodiment of the method of the invention for the parallel sequencing of nucleic acids by enzymatic strand extension thus comprises the following steps:
(i) Providing a monolithic porous support, having at least two sample chambers extending through the porous support, which have at least an inlet and an outlet and which have one or more surfaces to which nucleic acid molecules are immobilized having a doublestranded and a singlestranded portion, the porous support possessing at least two distinguishable sites, having nucleic acids possessing different sequences,
(ii) Providing a solution which has one or more nucleotides and a strandextending enzyme,
(iii) Introduction of the solution of step (ii) into the sample chambers of the porous support, in the course of which the enzyme, with formation of hydrogen bonds, binds the nucleotides to the immobilized nucleic acid molecules' singlestranded portions and, thus, indirectly to the porous support and incorporates them, at the boundary between doublestranded and singlestranded portion, into the immobilized nucleic acid molecules,
(iv) Detection of the amount and/or the identity of the nucleotides indirectly bound, via the immobilized nucleic acids, to the porous support, at the porous support's at least two distinguishable sites.

Where appropriate, step (ii) and the following steps are repeated, and specifically with in each case a different nucleotide than during the preceding step (ii).

Preferably, the sequencing of the nucleic acid molecules by enzymatic strand elongation is carried out according to one of the methods to be described now.
A) Incorporation of nucleotide triphosphates (“normal” nucleotides, dNTPs) upon determination of reaction side products,
B) Incorporation of labeled nucleotides,
C) Incorporation of reversibly labeled nucleotides,
D) Incorporation of labeled reversible chain terminating nucleotides.

A) In a preferred embodiment of the beforementioned method of the invention, the solution in step (ii) contains only one of the four nucleotides dATP, dGTP, dCTP and dTTP, and step (iv) comprises the detection of the amount of nucleotides indirectly bound to the porous support via the immobilized nucleic acids by determining the amount of formed reaction side-products, and step (iv) is followed by a step (v), comprising the removal from the porous support of non-incorporated nucleotides and, where appropriate, of reaction side-products, if step (ii) and the following steps are repeated, and specifically with in each case a different nucleotide than during the preceding step (ii).

Upon sequencing according to method (A), the formation of pyrophosphate connected with the incorporation of nucleotide triphosphates into the growing strand can be measured, as described by Ronaghi et al. (Analyt. Biochem. 242, 84-89 [1996]). In this connection, the generated pyrophosphate is converted, by means of sulfurylase, into ATP, which in turn participates in a luciferase-catalyzed chemoluminescence reaction and thus can be detected. According to this method, with the aid of the flow through arrangement, at each sequencing cycle a solution is flowing through the hollow spaces or channels of the porous support, the solution containing a particular desoxynucleotide triphosphate, e.g., dATP or its thio-analog dATPaS, as well as further components required for strand extension such as polymerase and, where appropriate, buffer, ions, etc. The amount of pyrophosphate set free upon a particular nucleotide flowing through the sample chambers corresponds to the amount of ATP generated. This is determined luminometrically, resolved by position. Thus, it can be determined at which sites of the porous support in each case incorporation of a nucleotide into the growing nucleic acid strand has occurred. After removal of excess, non-incorporated dATP by washing (Ronaghi et al., 1996) or by degradation by apyrase (Ronaghi et al., Science 281, 363-365 [1998]), in the next sequencing cycle, a second specified nucleotide triphosphate, e.g., dCTP, is offered in the flow through solution and, again, the amount of formed ATP is determined. Non-. incorporated dCTP is removed, usually by a washing solution flowing through the porous support's sample chambers, and the procedure described above is repeated with a third and a fourth nucleotide triphosphate. Then the next reaction cycle is performed, consisting of the staggered addition of one nucleotide triphosphate at a time, the measurement of the amount of ATP formed, and the removal of non-incorporated nucleotide triphosphate, and this is repeated as often as desired. From the relative signal intensities, it can be determined for each addition of a nucleotide triphosphate whether, in the course of a strand elongation, one or more identical nucleotide bases per strand have been incorporated into the growing strand, such that from the sequence and intensity of the obtained chemoluminescence signals, resolved by position, the base sequence of the nucleic acid strand, the template, at the respective site of the porous support can be reconstructed. In this way, the sequences of the nucleic acids at different sites of the porous support can be identified and the base sequence within the sequenced portion of the sequence can be determined.

B) In another embodiment of the method of the invention, the solution in step (ii) contains only one of the four nucleotides dATP, dGTP, dCTP and dTTP, respectively, which each are labeled by a labeling group, and step (iii) is followed by a step (iii-b), which comprises removal of non-incorporated nucleotides from the porous support, and in step (iv) detection is carried out of the amount and/or the identity of the nucleotides indirectly bound to the porous support, by means of the immobilized nucleic acids, by determination of the amount and/or the identity of labeling groups bound to the support, at at least two distinguishable sites of the porous support. Where appropriate, step (ii) and the following steps are repeated, and specifically with in each case a different nucleotide than in the preceding step (ii).

Upon the sequencing according to method (B), a nucleic acid molecule to be sequenced is incubated, under conditions favorable to a polymerase-catalyzed fill-in reaction, with one sort of labeled nucleotide at a time, thus, with labeled dATP, dCTP, dGTP, or dTTP, respectively. This is carried out as already described for method (A) by, during each sequencing cycle, a solution flowing through the porous support's sample chambers, the solution containing a particular nucleotide, the enzyme, as well as, where appropriate, further components permitting strand elongation. After removal of non-incorporated nucleotides, usually by a washing solution flowing through the porous support, it is determined by means of detection of the label, resolved by position, whether and how many nucleotides have been incorporated, respectively (e.g., 1×A, 2×A, etc.), and within which sites of the porous support this took place. In the next step, it is incubated with a second sort of labeled nucleotide (e.g., labeled dCTP) and detected, then the same is performed with a third (e.g., labeled dGTP) and finally with a fourth sort of nucleotide (e.g., labeled dTTP). Then, the cycle begins again by adding labeled nucleotide of the first sort. The signal intensities measured upon a detection result in each case from the sum of the signal intensities resulting from the nucleotide incorporation performed last and all previous nucleotide incorporations.

From the relative signal intensities, for each addition of a nucleotide triphosphate it can be determined, whether in the course of strand extension within a cycle one or more identical nucleotide bases per strand have been incorporated into the growing strand, such that from the sequence and intensity of the obtained label signals, resolved by position, the base sequence of the nucleic acid molecule at the respective site of the porous support (thus, resolved by position) can be reconstructed. In this way, different areas or distinguishable sites of nucleic acids having identical sequences on the porous support can be identified and the base sequence within the sequenced sequence portion can be determined.

C) In a particular embodiment of the method of the invention, the nucleotides are reversibly labeled, and, for reduction of the signal background, the label of nucleotides already incorporated is deleted after passing through steps (ii) to (iv) once or several times.

According to method (C), the incorporation of reversibly labeled nucleotides is carried out according to method (B), however, here the incorporated nucleotides' label is deleted at appropriate time points, e.g., after each addition of nucleotides or after each cycle, consisting of consecutive addition of all four nucleotides, or one or more repetitions of the cycle, respectively. Preferably this occurs by removal or alteration of the labeling group or labeling groups. For example, the labeling group can be linked to the respective nucleotide by a chemically, photochemically or enzymatically cleavable spacer, e.g., a spacer containing a disulfide group or a nitrobenzyl group, which is cleaved off photochemically or chemically. If enzymatic cleavage has been chosen, preferably this occurs by a solution containing the cleaving enzyme flowing through the porous support's hollow spaces or channels, with the aid of the flow through arrangement. Attachment of the labeling group at the nucleotide's nucleobase is well suited. The photochemical cleavage occurs by excitation of the cleavable group by light of appropriate intensity and wave length with the aid of a laser, for example. One possibility to alter the labeling group would be, e.g., bleaching of a fluorescent dye, which would be possible, e.g., by sufficiently intense irradiation by a laser. The advantage of method (C) over (B) consists in that, upon measurement, only a part in each case of the incorporated nucleotides is determined, ideally exclusively the nucleotide incorporated last, without the need to consider the signal background caused by the nucleotides already incorporated previously, which may be several times that of the signal of interest.

D) In another embodiment of the method of the invention, the solution of step (ii) contains one or more nucleotides, selected from dATP, dGTP, dCTP and dTTP, the nucleotides comprising a removable group which causes chain termination and has a labeling group, and step (iii) is followed by a step (iii-b) comprising the removal of non-incorporated nucleotides from the porous support, and in step (iv) the detection of the amount of nucleotides indirectly bound to, by means of the immobilized nucleic acids, the porous support occurs by determining the amount of labeling groups bound to the support at at least two distinguishable sites of the porous support, and between step (iii-b) and step (iv) or between step (iv) and step (v), step (vi) is executed, comprising the removal of the group causing chain termination from the nucleotides bound to the porous support.

Thus, this preferred embodiment of the method of the invention for the parallel sequencing of nucleic acids by enzymatic strand extension comprises the following steps:
(i) Providing a monolithic porous support, comprising at least two sample chambers extending through the porous support, which has at least an inlet and an outlet and which have one or several surfaces to which nucleic acid molecules having a doublestranded and a singlestranded portion are immobilized, the porous support possessing at least two distinguishable sites having nucleic acids having different sequences,
(ii) Providing a solution containing a strand extending enzyme and one or more nucleotides selected from dATP, dGTP, dCTP and dTTP, the nucleotides having a removable group causing chain termination and being labeled by a labeling group,
(iii) Introduction of the solution of step (ii) into the porous support's sample chambers, in the course of which the enzyme binds with formation of hydrogen bonds the nucleotides to the singlestranded portions of the immobilized nucleic acid molecules and, thus, binds them indirectly to the porous support, and incorporates them at the boundary between the immobilized nucleic acid molecules' doublestranded and singlestranded portion,
(iii-b) Removing the non-incorporated nucleotides from the porous support,
(iv) Detecting the amount and/or identity of the nucleotides indirectly bound to, by means of the immobilized nucleic acids, the porous support, by determination of the amount and/or identity of the labeling groups bound to the support at the at least two distinguishable sites of the porous support,
(v) Removing the group causing chain termination from the nucleotides indirectly bound to, by means of the immobilized nucleic acids, the porous support, in the course of which steps (iv) and (v) may be interchanged.

Preferably, the removable group causing chain termination represents, at the same time, the labeling group, and steps (iv) and (v) are not interchanged.

The sequencing according to method (D) by incorporation of reversible chain terminating nucleotides may be carried out as described in U.S. Pat. No. 5,302,509, U.S. Pat. No. 5,798,210, or WO 01/48184, which are hereby fully incorporated as reference, for example. When the procedure is according to method (D), labeled nucleotides as described for method (B) can be employed as well as reversibly labeled nucleotides as described for method (C), however, the nucleotides having the additional feature of possessing a functional group causing chain termination which, however, can be removed. According to method (D), the nucleotide-wise elongation of nucleic acid strands is accomplished by using nucleotide triphosphates reversibly blocked at their 3′-OH group, which can be incorporated by polymerases into a growing DNA doublestrand, but which, after their incorporation, act as chain elongation terminators. When the blocking group is cleaved off, a free 3′-OH group is restored, such that a next nucleotide can be incorporated. For example, Canard and Sarfati (Gene 148, 1-6 [1994]) describe reversibly blocked nucleotide triphosphates which can be identified, by means of the fluorescence label of the reversible protecting group, after their incorporation. In method (D), in each sequencing cycle a solution is flowing through the porous support s sample chambers, the solution containing a particular desoxynucleoside triphosphate or more, particularly all four desoxynucleoside triphosphates. Subsequently, the removal of non-incorporated nucleotides is carried out, usually by flowing a washing solution through the porous support, and the determination, resolved by position, of the location on the porous support at which a particular nucleotide has been incorporated. Thus, it is possible to track, by measuring, the incorporation of one single nucleotide per sequencing cycle, in the course of which the information for all four sorts of nucleotide can be obtained simultaneously, provided the solution used for flowing through the porous support's sample chambers contained all four nucleotides. Offering all four sorts of nucleotides at the same time is possible due to the fact that, because of their function as chain terminators, always only one nucleotide can be incorporated per cycle, until the blocking group is cleaved off again, which may be carried out chemically, photochemically or enzymatically (see WO 01/48184) and which may occur before or after the determination of the site on the porous support, resolved by position, where a particular nucleotide had been incorporated. Herewith, per sequencing cycle comprising step (ii) to step (v), only one single nucleotide is incorporated, other than in methods (A) to (C) mentioned before, where as many nucleotides would be to be incorporated into the growing DNA strand of an immobilized nucleic acid, until a nucleotide would have to be incorporated which is not offered by the solution of step (ii) and (iii) during the respective sequencing cycle. Herewith, it is possible to offer all nucleotides simultaneously in one sequencing cycle in step (iii), provided the nucleotides are distinguishable as a result of different labels. For longer read lengths, it can be expedient to employ reversibly labeled nucleotides to drive back the background caused by already incorporated nucleotides. Removal of the labels may be carried out after one or several sequencing cycles, depending on which background seems to be still tolerable, which, among other things, depends on the read length. However, preferably the removable group causing chain termination is, at the same time, the labeling group, consequently the labeling group doesn't have to be deleted or removed separately. In this case, steps (iv) and (v) are not interchanged.

The described methods for sequencing the nucleic acid molecules by enzymatic strand elongation (A) to (D) are based on the employment of enzymes, usually DNA polymerases, which elongate a DNA singlestrand complementary to the DNA strand to which the DNA singlestrand is bound. This requires that the nucleic acid molecules in step (i) have a singlestranded and a doublestranded portion. If the nucleic acids are not present in this state from the beginning, this state is produced in steps (i-a4) and (i-b3). If the amplification is carried out by PCR and the immobilization of the nucleic acid molecules is carried out via primers immobilized to the sample chambers' surfaces, immobilization of only one primer of a primer pair is advantageous since, in this way, only one nucleic acid strand of a doublestranded nucleic acid molecule is immobilized, thus facilitating removal of the non-immobilized nucleic acid strand, which provides nucleic acid molecules with a singlestranded portion.

To allow for sequence determination by incorporation of nucleotide building blocks into a growing strand according to the known base pairing rules, in one embodiment a sequencing primer is employed, i.e., an oligo- or polynucleotide able to hybridize with the nucleic acid strand to be sequenced and which, in the hybridized state, is capable of being elongated, by a DNA polymerase, at its 3′-end, in the course of which the opposite strand complementary to the region to be sequenced is synthesized (intermolecular priming of the polymerase). Accordingly, where appropriate, an immobilized doublestranded nucleic acid molecule is converted, by partial or complete removal of the opposite strand, to a state in which it has a singlestranded portion, and then an appropriate sequencing primer which is at least partially complementary to the nucleic acid molecule and which has a 3′-end that can be elongated by a polymerase is hybridized to the nucleic acid molecule, such that the nucleic acid molecule now is in a state in which it has a singlestranded and a doublestranded portion.

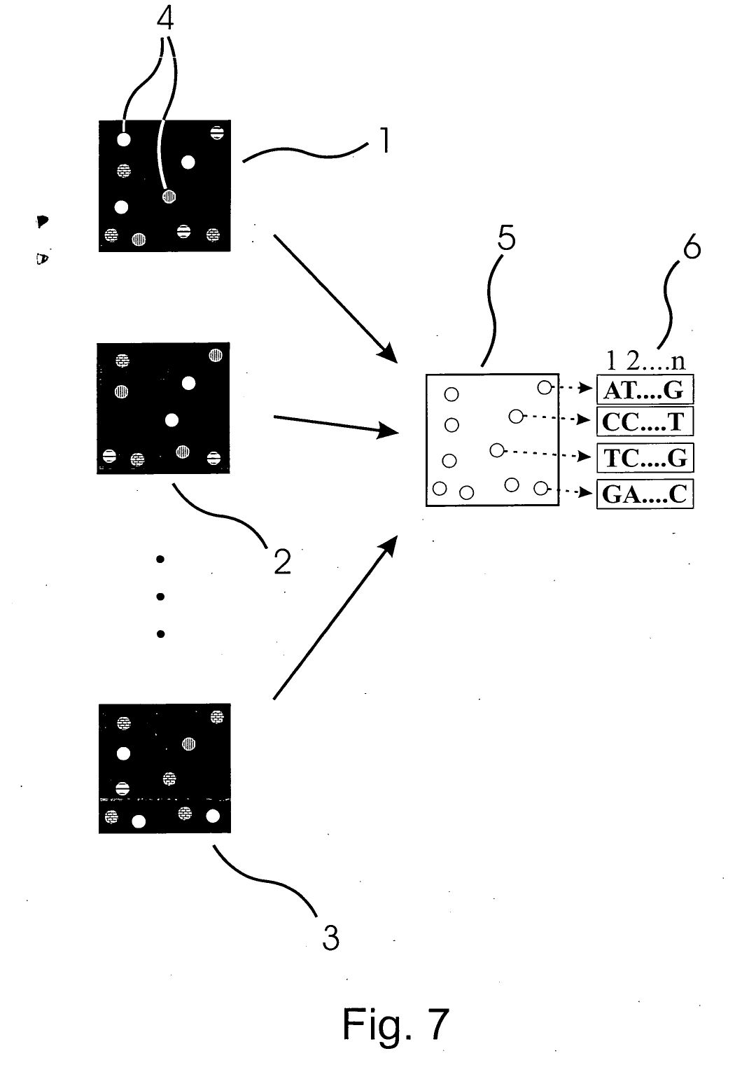

In an alternative embodiment, upon amplification of the nucleic acid molecules by PCR, a primer is employed having self-complementary regions (see WO 01/48184, page 9, first bullet point), such that the PCR-amplified nucleic acid molecules, after partial or complete denaturation with partial or complete removal of the opposite strand, fold back with hairpin formation, thus generating singlestranded and doublestranded portions. Moreover, as described, e.g., on page 4, line 63 ff., and in FIG. 7 of the U.S. Pat. No. 5,798,210, a hairpin structure can be attached to a nucleic acid molecule having a singlestranded portion, in the course of which singlestranded portions and doublestranded portions are generated as well (intramolecular priming of the polymerase).

It is also possible to attach to the nucleic acid molecule in the doublestranded state even before the immobilization a “masked hairpin”, i.e., a doublestranded nucleic acid molecule containing an inverted repeat. When, after immobilization, one of the two strands is removed by denaturation, the opposite strand remaining at the nucleic acid molecule to be sequenced and attached to it via its 5′-end can “fold back”, thus forming singlestranded and doublestranded portions, and be elongated at its free 3′-end by a polymerase (see WO 01/48184, page 9, second bullet point).

Otherwise, besides enzymatic strand elongation or shortening, the sequencing may be carried out according to other methods such as SBH (SBH, sequencing by hybridization; see Drmanac et al., Science 260 (1993), 1649-1652) as well. In this method, at each sequencing cycle a solution is flowing through the porous support's sample chambers, the solution containing labeled oligonucleotides having a known sequence plus, where appropriate, further compounds ensuring the correct hybridization of the oligonucleotides with sequence portions of the nucleic acid molecules to be sequenced complementary thereto. If the labels are different in each case, the solution can contain as many sorts of oligonucleotides having different sequences as can be distinguished by means of their labels.

Thus, another embodiment of the invention relates to a method for parallel sequencing of nucleic acids by hybridization, in the course of which the nucleotide compounds in step (ii) are one or more oligonucleotides having a labeling group which, with formation of hydrogen bonds, hybridize to the immobilized nucleic acid molecules in step (iii) such that the oligonucleotides are bound to the porous support, and in the course of which in step (iii) determination of the amount of oligonucleotides bound to the porous support is carried out by determination of the amount of labeling groups bound to the support at at least two distinguishable sites of the porous support. Thus, this embodiment of the method has the following steps:
(i) Providing a monolithic porous support, having at least two sample chambers extending through the porous support, having at least an inlet and an outlet and having one or more surfaces to which nucleic acid molecules are immobilized, said nucleic acid molecules having a singlestranded portion, in the course of which the porous support possesses at least two distinguishable sites having nucleic acids having different sequences,
(ii) Providing a solution containing one or more oligonucleotides having labeling groups,
(iii) Introduction of the solution of step (ii) into the porous support's sample chambers, in the course of which the oligonucleotides, with formation of hydrogen bonds, are bound to the singlestranded portions of the immobilized nucleic acid molecules and, thus, are bound indirectly to the porous support,
(iv) Detecting the amount and/or identity of the oligonucleotide compounds indirectly bound to, by means of the immobilized nucleic acids, the porous support, by determination of the amount of labeling groups bound to the support at the at least two distinguishable sites of the porous support.

The steps (ii) to (iv) can be repeated, in the course of which during each cycle sequence information is obtained.

The invention further relates to a monolithic porous, support having at least two sample chambers extending through the porous support, which have at least an inlet and an outlet and which possess one or more surfaces to which nucleic acid molecules are immobilized.

Preferably the nucleic acid molecules immobilized to the porous support have a singlestranded portion, and on the porous support there are at least two distinguishable sites possessing nucleic acids having different sequences.

The invention further relates to a monolithic porous support, having at least two sample chambers extending through the porous support, which have at least an inlet and an outlet, and which possess one or more surfaces,. the surfaces having a coating appropriate to immobilization of nucleic acids.

A further solution of the problem consists in a method for the parallel sequencing of nucleic acids, comprising the steps:
(vi) Providing a monolithic porous support, having at least two sample chambers filled by liquid, extending through the porous support and possessing at least an inlet and an outlet, the porous support having at least two distinguishable sites possessing nucleic acids having different sequences,
(vii) Amplifying the nucleic acid molecules in the sample chambers,
(viii) Providing a surface,
(ix) Contacting, with formation of a liquid film between the surface and the sample chambers, the surface of step (viii) with the porous support,
(x) Immobilizing the nucleic acids of step (ix) on the surface with formation of at least two distinguishable places on the surface, possessing nucleic acids having different sequences,
   in the course of which the steps (vii), (viii), (ix) and (x) may be carried out
   simultaneously, (xi) Converting the nucleic acids on the surface to a state in which they have a singlestranded portion, this step being able to take place between steps (vii) and (x) or at the same time of these steps as well,
(xii) Providing a solution containing one or more nucleotide compounds, selected from mono- and oligonucleotides,
(xiii) Contacting the solution of step (xii) with the nucleic acids on the surface of step (xi), in the course of which the binding of the nucleotide compounds to the immobilized nucleic acids' singlestranded portions and, thus, the indirect binding to the surface is effected,
(xiv) Detection of the amount and/or identity of the nucleotide compounds indirectly bound, by means of the immobilized nucleic acids, to the surface, at the at least two distinguishable places of the surface.

In a preferred embodiment of the method of the invention, step (vi) comprises the steps
(vi-a0) Providing a monolithic porous support, comprising at least two sample chambers extending through the porous support, which have at least an inlet and an outlet,
(vi-a1) Soaking of the porous support with a nucleic acid solution containing at least two nucleic acid molecules having different sequences, such that at least two sample chambers are filled with the nucleic acid solution, such that afterwards the porous support has at least two distinguishable sites which possess nucleic acids having different sequences.

In a further preferred embodiment of the method of the invention, step (vi) comprises the steps
(vi-b0) Providing a monolithic porous support, comprising at least two sample chambers extending through the porous support and having at least an inlet and an outlet,
(vi-b1) Transfer of at least two nucleic acid solutions containing in each case nucleic acid molecules having different sequences, to in each case different positions of the porous support, such that at least two sample chambers are filled with the nucleic acid solutions, such that afterwards the porous support has at least two distinguishable sites possessing nucleic acids having different sequences.

The monolithic porous support of step (vi) refers to the porous support of step (i), which has at least two sample chambers as well, extending through the porous support and having at least an inlet and an outlet. In this respect reference is made to what has been said above. However, the sample chambers are filled by liquid, since otherwise neither the amplification in step (vii) nor the formation of a liquid film in step (viii) will occur. The porous support thus has at least two distinguishable sites possessing nucleic acids having different sequences. Concerning the term “sites”, what has been said in step (i) is valid as well. In step (ix), the sites are transferred to a surface, in a way projected to a plane formed by the surface. After immobilization of the nucleic acids reference is made to places on the surface, possessing nucleic acids having different sequences. The nucleic acids in step (vi) don't have to possess singlestranded portions, and they don't have to be immobilized. However, of course the latter is not excluded as long as the immobilization is carried out reversibly or relates to only a single strand of a nucleic acid doublestrand, such that at least the respective opposite strand can be detached by denaturation and transferred in step (ix).

The amplification is carried out as described in (i-b), and in particular with the aid of PCR. Then, a primer of a primer pair or a portion of this primer could be immobilized to the sample chambers' surfaces as well. Thus, in the case of immobilized nucleic acids, the opposite strand of the amplification product can be detached by denaturation.

In step (viii), a surface is provided. This refers to the accessible surface of a body made from plastic, metal, glass, silicon, or similarly appropriate materials, which allow for immobilization of nucleic acids and, where appropriate, are functionalized accordingly. The surface can have a layer able to swell, e.g., made from polysaccharides, polysugar alcohols or silicates able to swell. In a special case, the surface represents a porous support as defined in step (vi).

Step (ix) comprises the contacting of the surface of step (viii) with the porous support. This can take place by simply placing the surface on the porous support. Since the sample chambers are filled by liquid, a liquid film is formed between the surface and the sample chambers. Within the scope of the invention, the term “filled by liquid” means the partial or complete occupation of a sample chamber's lumen. By this measure, after immobilization in the following step at least two distinguishable places on the surface are formed, possessing nucleic acids having different sequences. The nucleic acids having different sequences are transferred to the surface by diffusion or convection. The latter plays a part particularly if the surface is formed by a porous support able to suck in or absorb the liquid in the sample chambers by capillary forces. Step (ix) results, on the surface, in a projection of the porous support's distinguishable sites to a plane, whereby, after immobilization, distinguishable places on the surface are obtained.

With respect to the immobilization of the nucleic acid molecules in step (x), what has been said concerning step (i-a3) or (i-b2) is valid accordingly.

Usually, converting the nucleic acids in step (xi) to a state in which they have a singlestranded portion is carried out by full or partial denaturing of a nucleic acid doublestrand and, where appropriate, removal of the opposite strand. This step may be carried out between steps (vii) and (x) or at the same time of these steps as well. However, the converting should be facilitated if the nucleic acids have been immobilized already. Reference may be made to the steps (i-a4) and (i-b3). Preferably, in step (xi) nucleic acids are converted to a state in which they have a singlestranded portion. This is especially the case when the sequencing is carried out by enzymatic strand elongation.

Concerning steps (xii), (xiii) and (xiv), reference may be made to the steps (ii), (iii) and (iv) accordingly, in the course of which, however, in step (xiii) the liquid provided in the previous step is only contacted with the surface. However, this does not exclude that the solution is introduced into the sample chambers, if the surface is formed by a porous support having sample chambers.

The steps (xii) to (xiv) can be repeated once or several times, in the course of which in each cycle sequence information is obtained.

In step (xiv) detection occurs whether nucleotide compounds have been bound indirectly to the surface. If the solution in step (ii) contains several nucleotide compounds, it is tested in step (xiv) which is the nucleotide compound that has been bound to the surface, i.e., their identity is determined. Usually it is also necessary to measure the amount of bound nucleotide compounds to be able to discriminate between significant signals and background. Under certain circumstances it is expedient to measure the amount more precisely. This is the case when, possibly, several nucleotide compounds can be bound to the immobilized nucleic acids' singlestranded portions in step (xiii) and this allows conclusions about the sequence to be drawn, such as is the case upon sequencing by enzymatic strand extension by nucleotides without chain termination group.

In a preferred embodiment of the method of the invention, the steps (xii), (xiii) and (xiv) are realized as follows:
(xii) Providing a solution containing one or more nucleotides and a strand extending enzyme,
(xiii) Contacting the solution of step (xii) with nucleic acids on the surface from step (xi), the enzyme binding, upon formation of hydrogen bonds, the nucleotides to the immobilized nucleic acid molecules' singlestranded portions and, thus, indirectly to the surface, and incorporating them, at the boundary between doublestranded and singlestranded portion, into the immobilized nucleic acid molecules,
(xiv) Detection of the amount and/or the identity of nucleotides bound indirectly to, by means of the immobilized nucleic acids, the surface, at the at least two distinguishable places on the surface.

Where appropriate, step (xii) and the subsequent steps are repeated, and specifically with in each case a different nucleotide than at the previous step (ii).

The sequencing of the nucleic acid molecules by enzymatic strand extension preferably is carried out by one of the methods to be-described now.
B) Incorporation of labeled nucleotides,
C) Incorporation of reversibly labeled nucleotides,
D) Incorporation of labeled reversible chain terminating nucleotides.

Concerning these methods, reference is made to what has been said with regard to steps (ii) to (xiv).

B) In one embodiment of the method of the invention, the solution in step (xii) contains only one of the four nucleotides dATP, dGTP, dCTP and dTTP, respectively, which in each case are labeled by a labeling group, and step (xiii) is followed by a step (xiii-b) comprising removal of non-incorporated nucleotides from the surface, and in step (xiv) detection takes place of the amount and/or identity of the nucleotides bound indirectly, by means of the immobilized nucleic acids, to the surface, by determination of the amount and/or the identity of the labeling groups bound to the surface, at at least two distinguishable places on the surface. Where appropriate, step (xii) and the subsequent steps are repeated, and specifically with in each case a different nucleotide than in the previous step (xii).

C) In a particular embodiment of the method of the invention, the nucleotides are reversibly labeled, and, for reduction of the signal background, the labeling of nucleotides already incorporated is deleted after one or repeated passing through of steps (xii) to (xiv).

D) In a preferred embodiment of the method of the invention, the steps (xii), (xiii) and (xiv) are realized as follows:
(xii) Providing a solution containing a strand extending enzyme and one or more nucleotides, selected from dATP, dGTP, dCTP and dTTP, the nucleotides having a removable group causing chain termination and being labeled with a labeling group,
(xiii) Contacting the solution of step (xii) with the nucleic acids on the surface of step (xi), the enzyme binding, with formation of hydrogen bonds, the nucleotides to the immobilized nucleic acid molecules' singlestranded portions and, thus, indirectly to the surface, and incorporates them at the boundary between doublestranded and singlestranded portion into the immobilized nucleic acid molecules,
(xiii-b) Removal of non-incorporated nucleotides from the surface,
(xiv) Detection of the amount and/or the identity of the nucleotides bound indirectly to, by means of the immobilized nucleic acids, the surface, by determining the amount and/or the identity of labeling groups bound to the surface, at the at least two distinguishable places on the surface,
(xv) Removal of the group causing chain termination from the nucleotides bound indirectly to, by means of the immobilized nucleic acids, the surface, in the course of which steps (xiv) and (xv) may be interchanged.

Preferably the removable group causing chain termination represents, at the same time, the labeling group, and steps (xiv) and (xv) are not interchanged.

In another embodiment of the method of the invention, steps (xii), (xiii) and (xiv) are realized as follows:
(xii) Providing a solution containing one or more oligonucleotides having labeling groups,
(xiii) Contacting the solution of step (xii) with the nucleic acids on the surface of step (xi), in the course of which, with formation of hydrogen bonds, the oligonucleotides are bound to the immobilized nucleic acid molecules' singlestranded portions and, thus, are bound indirectly to the surface,
(xiv) Detection of the amount and/or identity of the oligonucleotide compounds bound indirectly to, by means of the immobilized nucleic acids, the surface, by determining the amount of labeling groups bound to the surface, at the at least two distinguishable places on the surface.

The invention is described in more detail by the drawings, showing the following:

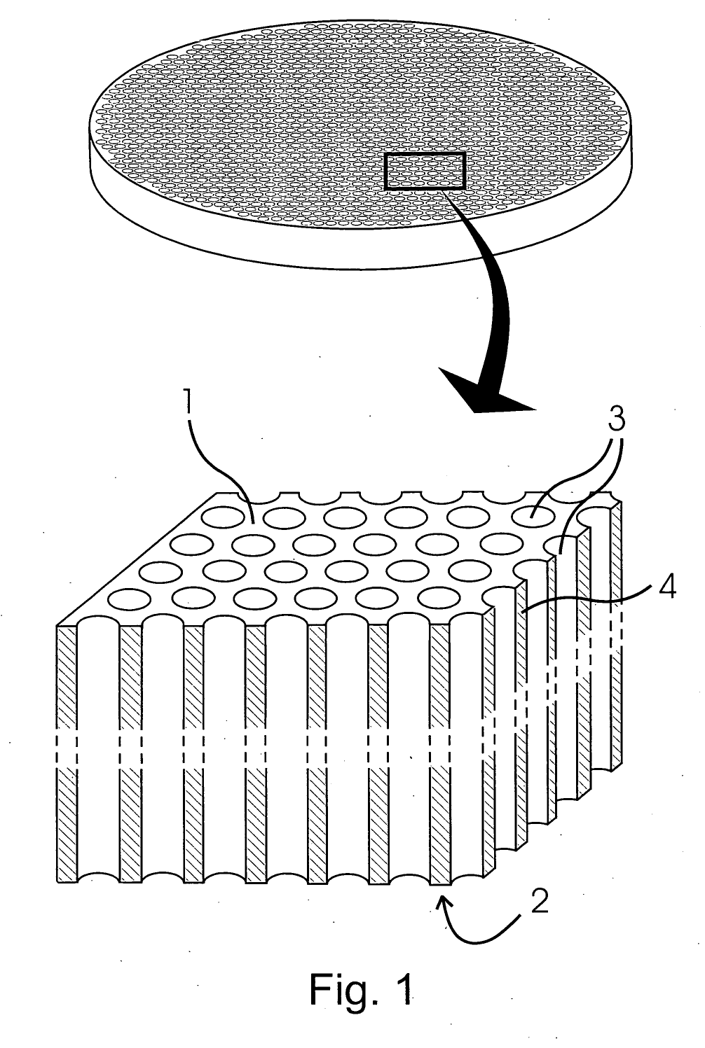

FIG. 1 a porous support for carrying out the method of the invention;

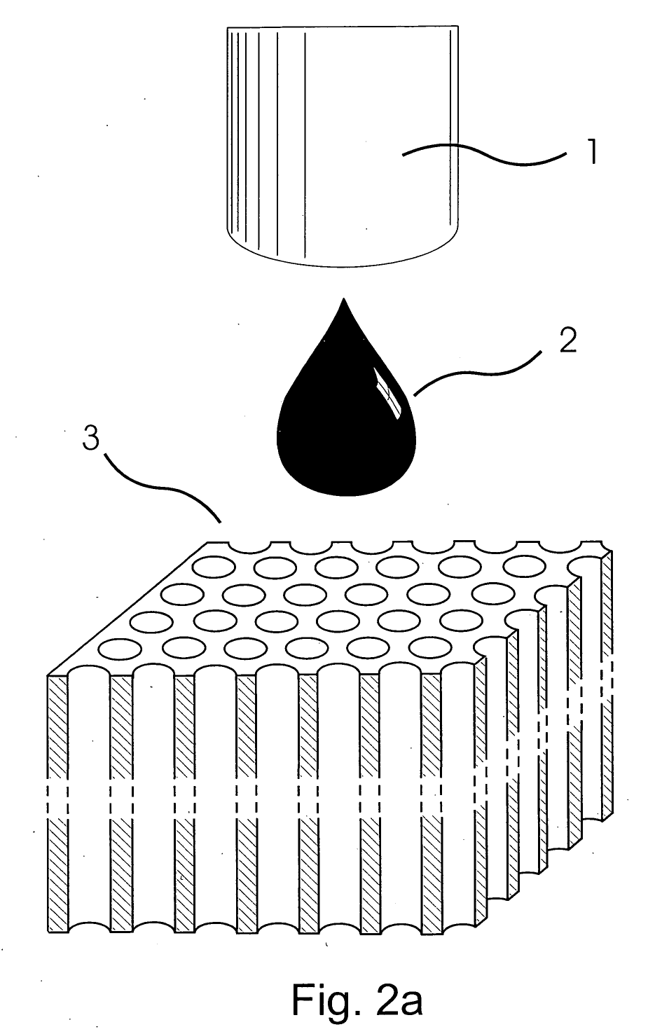

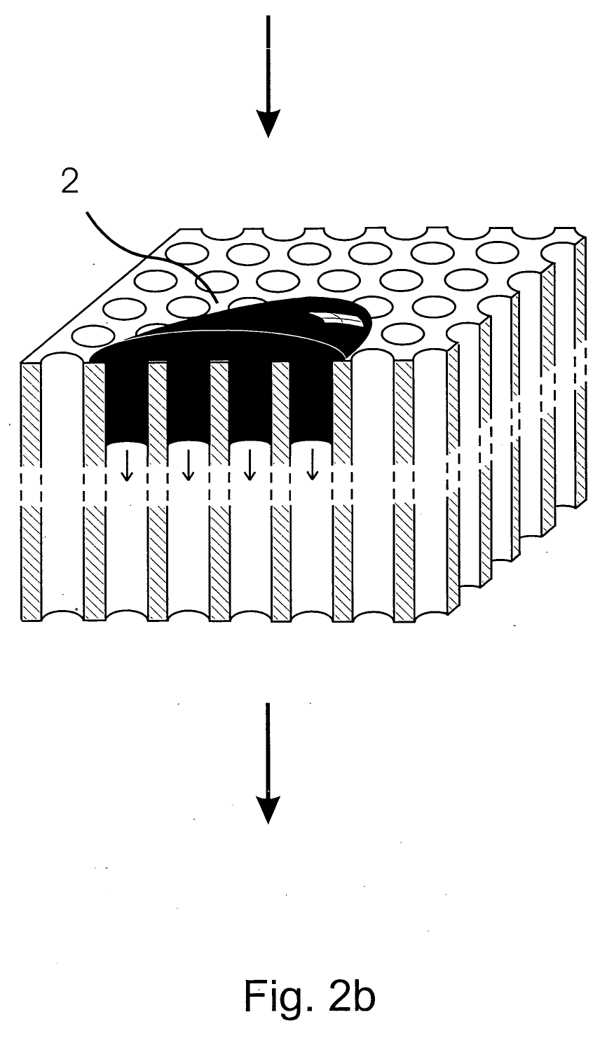

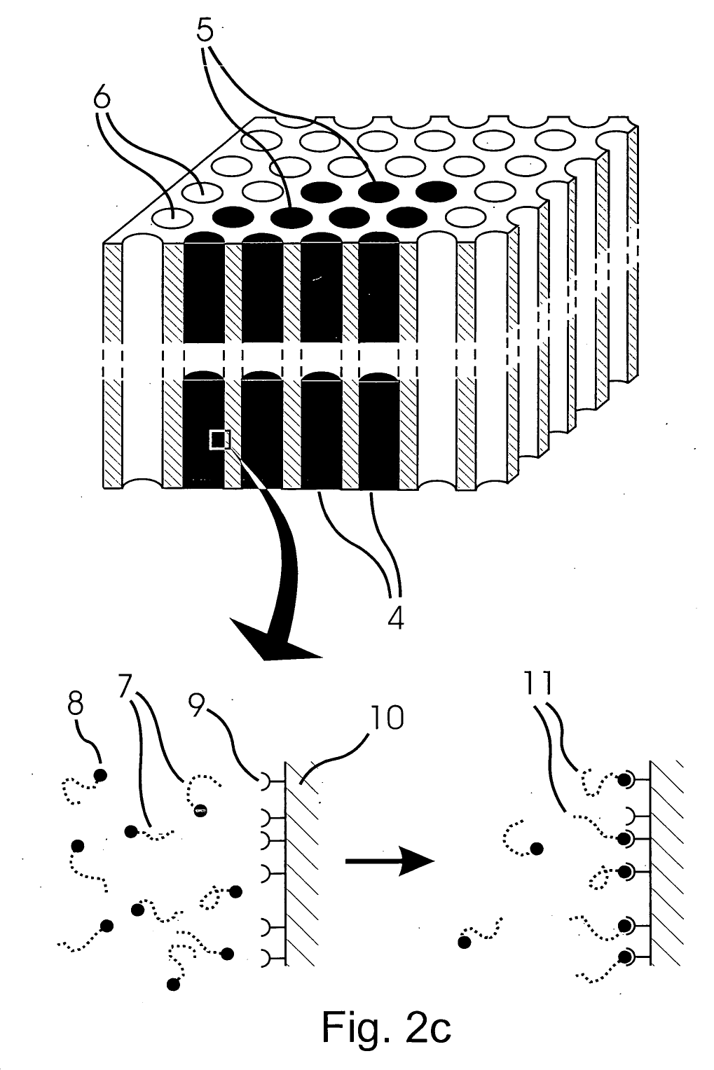

FIG. 2 the immobilization of nucleic acids within the porous support's channels;

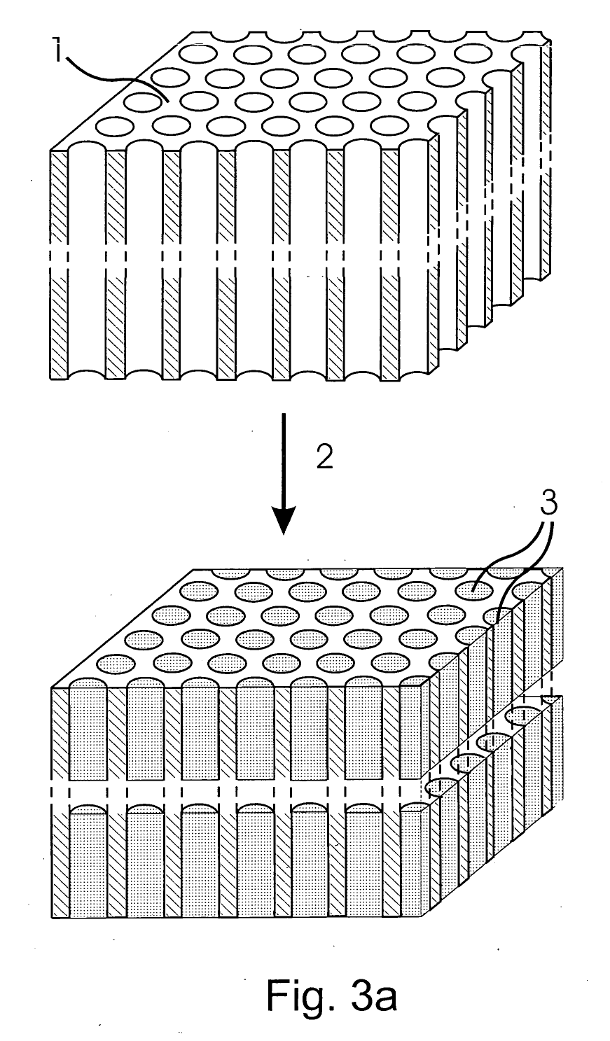

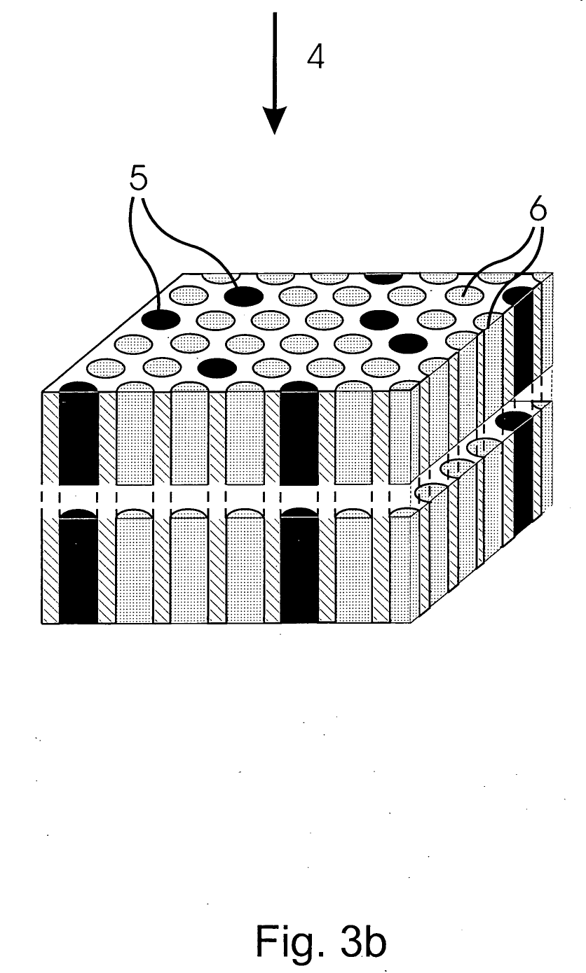

FIG. 3 the amplification of an appropriately diluted solution of nucleic acid molecules in the porous support s channels;

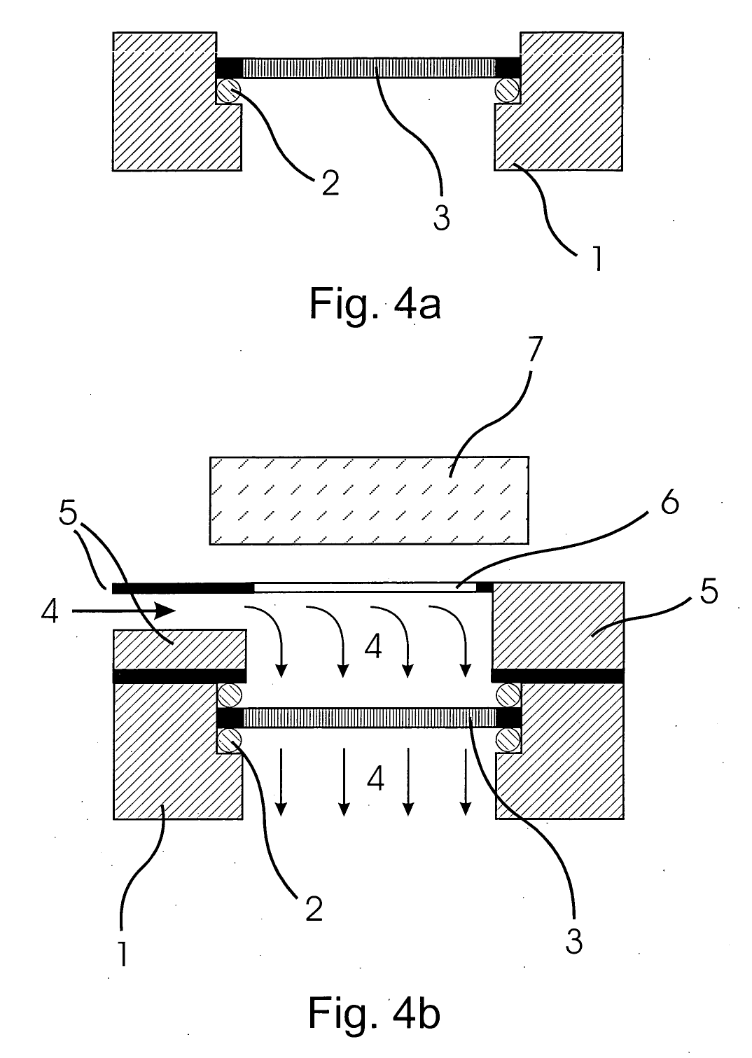

FIG. 4 a flow through arrangement for carrying out the method of the invention;

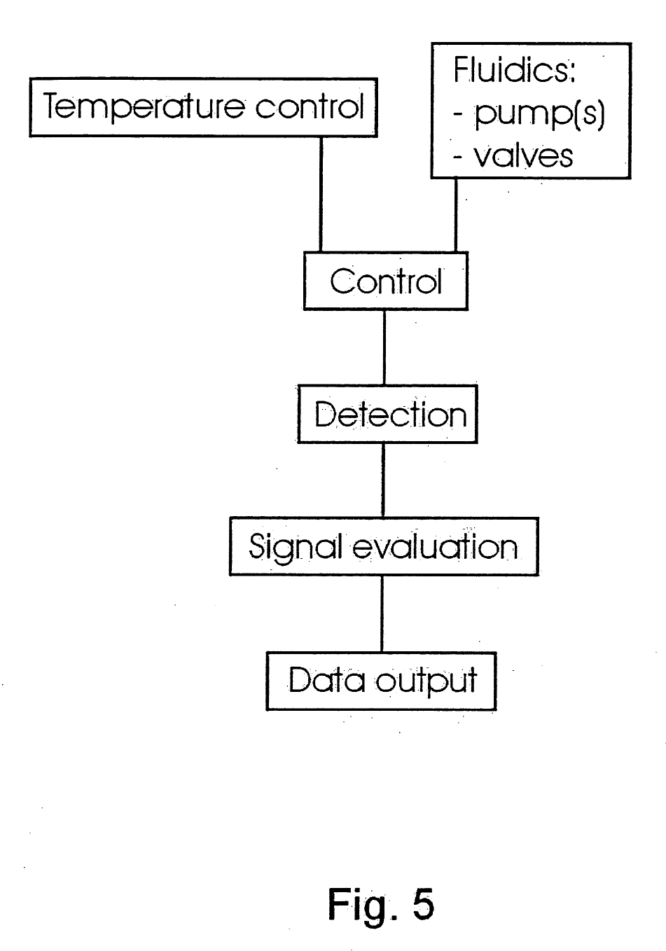

FIG. 5 a possible function structure for the flow through arrangement of FIG. 4;

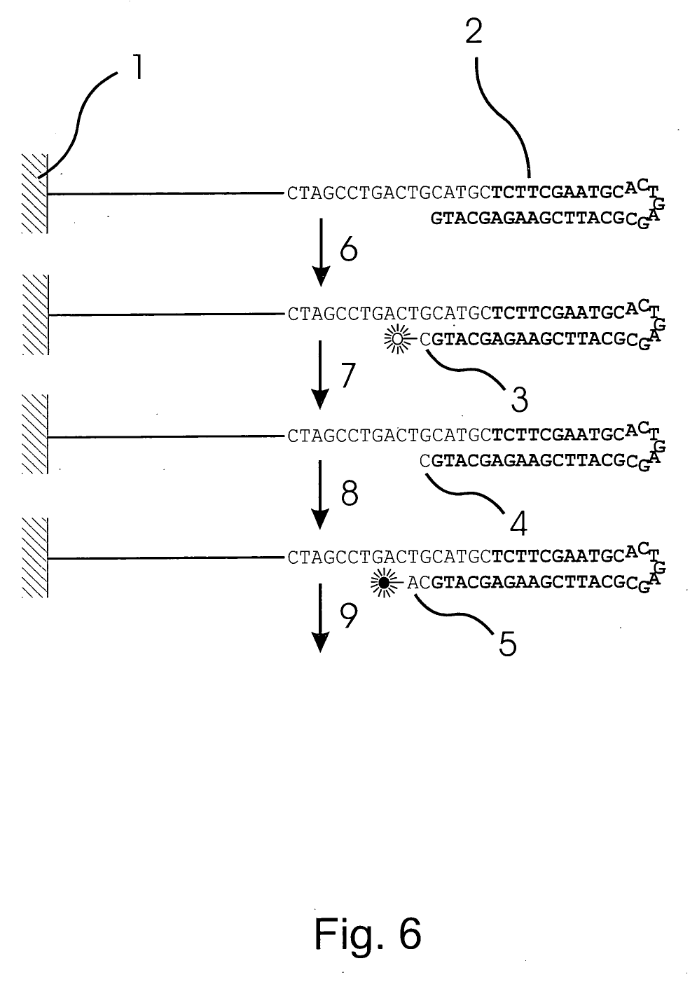

FIG. 6 the sequencing of immobilized nucleic acid molecules by means of reversible chain terminating nucleotides;

FIG. 7 the simultaneous sequencing of the immobilized nucleic acid molecules in several different areas of the porous support.

FIG. 1 indicates a porous support for carrying out the method of the invention, with
1 the porous support's top side,
2 the porous support's bottom side,
3 channels running through from the porous support's top side to its bottom side,
4 a partition between two adjoining channels.

FIG. 2 shows the immobilization of nucleic acids within the porous support's channels, with
1 a device for transferring the nucleic acid solutions to the porous support, such as, e.g., a pin, a capillary, or a jet,
2 a desired volume of a solution of nucleic acid molecules of the same sort, which due to the action of capillary forces is absorbed, after transfer to the porous support's top side, by the support's channels,
3 a detail of the porous support,
4 a solution of nucleic acid molecules absorbed, by means of capillary forces, by the porous support's channels,
5 filled channels,
6 non-filled channels,
7 dissolved nucleic acid molecules of the same sort,
8 an atom group mediating the terminal irreversible immobilization of a nucleic acid molecule (one partner of a specific binding pair),
9 an atom group capable of specifically binding to the atom group (8) (the other partner of the same specific binding pair),
10 a channel's wall,
11 nucleic acid molecules of the same sort, immobilized to the channel's wall.

FIG. 3 depicts the amplification of an appropriately diluted solution of nucleic acid molecules in the porous support's channels, with
1 the porous support,
2 the filling of essentially all the support's channels by a diluted solution of different nucleic acid molecules in an amplification mixture, containing the reagents required for carrying out an amplification reaction,
3 channels filled by the solution of (2),
4 amplification of single molecules in those channels having contained, after filling, one amplifiable nucleic acid molecule each, to numerous copies of these molecules,
5 channels in which an amplification of one nucleic acid molecule to numerous copies has taken place,
6 channels in which no amplification of nucleic acid molecules has taken place.

FIG. 4 indicates a flow through arrangement for carrying out the method of the invention, with FIG. 4a: flow through arrangement after inserting the porous support and FIG. 4b: flow through arrangement in operation, with
1 holding device of the support,
2 O-seal,
3 porous support,
4 current of an appropriately tempered reagent solution for sequencing of the nucleic acid molecules immobilized to the porous support,
5 lid,
6 observation window,
7 detector.

FIG. 5 shows a possible function structure of the flow through arrangement of FIG. 4.

FIG. 6 shows the sequencing of immobilized nucleic acid molecules by means of reversible chain terminating nucleotides, with
1 a channel's wall,
2 a nucleic acid molecule having a terminal hairpin structure,
3 a C nucleotide, reversibly protected at its 3′position, by a fluorescently labeled protecting group, against further strand extension,
4 the C nucleotide of (3) after cleaving off the protecting group, upon restoration of a 3′OH group,
5 an A nucleotide, reversibly protected at its 3′end, by a fluorescently labeled protecting group, against further strand extension,
6 a strand extension by one base, made possible by the incorporation of a fluorescently labeled dCTP derivative, reversibly protected at its 3′position,
7 the detection, with identification of the nucleotide incorporated last, of the fluorescent label incorporated into the strand in step (6), followed by cleaving off the fluorescent protecting group,
8 a further strand extension by one base, enabled by incorporation of a fluorescently labeled dATP derivative reversibly protected at its 3′position,
9 repetition of the steps detection, cleaving off, and strand extension by one base, until the desired read length is achieved.

FIG. 7 shows the simultaneous sequencing of nucleic acid molecules immobilized to several different areas of the porous support, with
1 a detail of the support containing different areas, the signals obtained from the sequencing of a first base being identified by different filling patterns in the figure,
2 a detail of the support containing the same areas, the signals obtained from the sequencing of a second base being identified by different filling patterns in the figure,
3 a detail of the support containing the same areas, the signals obtained from the sequencing of an n^{th} base being identified by different filling patterns in the figure,
4 two different areas of the porous support, each comprising one or more channels,
5 a superimposition of the results obtained, upon sequencing the first base up to the n^{th} base, for all covered areas,
6 the sequencing results obtained in (5) for the first to the n^{th} base of the nucleic acid molecules immobilized to the covered areas.

1.-31. (canceled)

32. A method for the parallel sequencing of nucleic acids, comprising the steps:
(1) providing a porous support possessing areas distinguished by immobilized nucleic acid molecules;
(2) inserting the support of step (1) into a flow through arrangement; and
(3) simultaneously determining at least a part of the nucleotide sequence of at least part of the nucleic acid molecules.

33. The method of claim 32, wherein the porous support is formed to have two parallel even surfaces and has a top side and a bottom side.

34. The method of claim 33, wherein the porous support possesses channels which are essentially parallel to each other and via which the top side and the bottom side communicate with each other.

35. The method of claim 34, wherein the channels' diameter is between 0.5 μm and 50 μm.

36. The method of claim 35, wherein the channels' diameter is between 1 μm and 25 μm.

37. The method of claim 32, wherein the porous support is made from glass.

38. The method of claim 37, wherein the porous support is a glass capillary array.

39. The method of claim 32, wherein the porous support is made from silicon.

40. The method of claim 32, wherein the nucleic acid molecules being positioned in the areas of step (1) have been transferred to the support as preformed nucleic acid solutions.

41. The method of claim 40, wherein the transfer has been made by pins, capillaries, or ink jet technology.

42. The method of claim 32, wherein the nucleic acid molecules being positioned in the areas of step (1) have been generated by amplification within the support's hollow spaces.

43. The method of claim 42, wherein at the beginning of the amplification, there are on average at most 0.5 amplifiable nucleic acid molecules within a hollow space.

44. The method of claim 43, wherein at the beginning of the amplification, there are on average at most 0.2 amplifiable nucleic acid molecules within a hollow space.

45. The method of claim 43, wherein at the beginning of the amplification, there are on average between 0.1 and 0.02 amplifiable nucleic acid molecules within a hollow space.

46. The method of claim 42, wherein upon amplification at least 10⁶ copies of a starting molecule are generated.

47. The method of claim 46, wherein upon amplification at least 10⁷ copies of a starting molecule are generated.

48. The method of claim 47, wherein upon amplification at least 10⁸ copies of a starting molecule are generated.

49. The method of claim 32, wherein the sequencing of the nucleic acid molecules is carried out by incorporation of nucleotide triphosphates and by determination of reaction side products.

50. The method of claim 32, wherein the sequencing of the nucleic acid molecules is carried out by incorporation of labeled nucleotides.

51. The method of claim 32, wherein the sequencing of the nucleic acid molecules is carried out by incorporation of reversibly labeled nucleotides.

52. The method of claim 32, wherein the sequencing of the nucleic acid molecules is carried out by incorporation of labeled reversible chain terminating nucleotides.

53. The method of claim 32, wherein the support has at least 10³ areas.

54. The method of claim 53, wherein the support has at least 10⁴ areas.

55. The method of claim 54, wherein the support has at least 10⁵ areas.

56. The method of claim 55, wherein the support has at least 10⁶ areas.

57. The porous support of claim 33, wherein the walls have a coating appropriate for the immobilization of nucleic acid molecules.

58. The porous support of claim 53, wherein the walls have a coating appropriate for the immobilization of nucleic acid molecules.

59. The porous support of claim 33, comprising areas where in each case a plurality of essentially identical nucleic acid molecules is positioned.

60. The porous support of claim 53, comprising areas where in each case a plurality of essentially identical nucleic acid molecules is positioned.

61. The porous support of claim 33, comprising areas where in each case a plurality of essentially identical nucleic acid molecules is positioned, the areas at least in some cases consisting of a single hollow space each.

62. The porous support of claim 53, comprising areas where in each case a plurality of essentially identical nucleic acid molecules is positioned, the areas at least in some cases consisting of a single hollow space each.

63. A flow through arrangement comprising:
(a) at least two spaces connected by a porous support;
(b) a means for establishing a pressure difference;
(c) a detector;
(d) where appropriate, a source of radiation appropriate to fluorescence excitation, and
(e) where appropriate, containers for the storage of reagents for performing amplification reactions and/or sequencing reactions.

64. A method for the massively parallel sequencing of nucleic acids, comprising the steps:
(1) providing porous support;
(2) introducing to the porous support's hollow spaces an amplification mixture, containing amplifiable nucleic acid molecules;
(3) performing an amplification of the nucleic acid molecules in the support's hollow spaces;
(4) contacting the porous support with a surface, this step optionally having been carried out before performing the amplification;
(5) immobilization to the surface of at least a portion of the amplified nucleic acid molecules of step (3); and
(6) simultaneous determination of at least a portion of the nucleotide sequence of at least a portion of the nucleic acid molecules immobilized to the surface.

65. The method as claimed in claim 32, wherein after immobilization the porous support is removed from the surface.
